SEQUENCE LISTING (91 sequences)

SEQ ID NO: 1 - protein, 8 aa, Homo sapiens
GFTFSNYG

SEQ ID NO: 2 - protein, 8 aa, Homo sapiens
ISSDGRIK

SEQ ID NO: 3 - protein, 18 aa, Homo sapiens
AKDRFQFARSWYGDYFDY

SEQ ID NO: 4 - protein, 10 aa, Homo sapiens
VFSLSNARMG

SEQ ID NO: 5 - protein, 7 aa, Homo sapiens
IFSSDQK

SEQ ID NO: 6 - protein, 17 aa, Homo sapiens
ARINTAAYDYDSTTFDI

SEQ ID NO: 7 - protein, 8 aa, Homo sapiens
GFTLRTSS

SEQ ID NO: 8 - protein, 8 aa, Homo sapiens
ISNDGATK

SEQ ID NO: 9 - protein, 16 aa, Homo sapiens
ARETRHYSHGLNWFDP

SEQ ID NO: 10 - protein, 8 aa, Homo sapiens
GFTFSSFG

SEQ ID NO: 11 - protein, 8 aa, Homo sapiens
ISDEGRIK

SEQ ID NO: 12 - protein, 16 aa, Homo sapiens
ARDVKGHIVVMTSLDY

SEQ ID NO: 13 - protein, 8 aa, Homo sapiens
GFTFSSYA

SEQ ID NO: 14 - protein, 8 aa, Homo sapiens
ISYDGSTK

SEQ ID NO: 15 - protein, 19 aa, Homo sapiens
ATVSVEGYTSGWYLGTLDF

SEQ ID NO: 16 - protein, 8 aa, Homo sapiens
GFTFDYYA

SEQ ID NO: 17 - protein, 8 aa, Homo sapiens
ISWNSDNT

SEQ ID NO: 18 - protein, 20 aa, Homo sapiens
AKDISLVFWSVNPPRNGMDV

SEQ ID NO: 19 - protein, 8 aa, Homo sapiens
GFTFSSYG

SEQ ID NO: 20 - protein, 8 aa, Homo sapiens
ISFDGRNK

SEQ ID NO: 21 - protein, 17 aa, Homo sapiens
ARDDNLDRHWPLRLGGY

SEQ ID NO: 22 - protein, 8 aa, Homo sapiens
GFTFRNYA

SEQ ID NO: 23 - protein, 8 aa, Homo sapiens
ITSDGRNK

SEQ ID NO: 24 - protein, 20 aa, Homo sapiens
VTQRDNSRDYFPHYFHDMDV

SEQ ID NO: 25 - protein, 8 aa, Homo sapiens
GFSFSSYA

SEQ ID NO: 26 - protein, 8 aa, Homo sapiens
MSASGDST

SEQ ID NO: 27 - protein, 12 aa, Homo sapiens
ASPLRNYGDLLY

SEQ ID NO: 28 - protein, 8 aa, Homo sapiens
GFTFASYA

SEQ ID NO: 29 - protein, 8 aa, Homo sapiens
ISGGGGDT

SEQ ID NO: 30 - protein, 19 aa, Homo sapiens
ARLESATQPLGYYFYGMDV

SEQ ID NO: 31 - protein, 11 aa, Homo sapiens
QSLVYSDGNIY

SEQ ID NO: 32 - protein, 9 aa, Homo sapiens
MQGTHWPPT

SEQ ID NO: 33 - protein, 6 aa, Homo sapiens
QTISNY

SEQ ID NO: 34 - protein, 9 aa, Homo sapiens
QQSYSTPPT

SEQ ID NO: 35 - protein, 6 aa, Homo sapiens
QSVSSD

SEQ ID NO: 36 - protein, 9 aa, Homo sapiens
QQYNNWPTT

SEQ ID NO: 37 - protein, 11 aa, Homo sapiens
QSLVSSDGDTS

SEQ ID NO: 38 - protein, 9 aa, Homo sapiens
MQGTHWPPT

SEQ ID NO: 39 - protein, 6 aa, Homo sapiens
QSVSSN

SEQ ID NO: 40 - protein, 9 aa, Homo sapiens
QQYNNWPGT

SEQ ID NO: 41 - protein, 6 aa, Homo sapiens
QSIRSY

SEQ ID NO: 42 - protein, 9 aa, Homo sapiens
QQSYSSPLT

SEQ ID NO: 43 - protein, 11 aa, Homo sapiens
QDLLYNDGGTD

SEQ ID NO: 44 - protein, 9 aa, Homo sapiens
MQGAHWPPT

SEQ ID NO: 45 - protein, 11 aa, Homo sapiens
QSLVYSDGDTY

SEQ ID NO: 46 - protein, 9 aa, Homo sapiens
MQGSHWPPT

SEQ ID NO: 47 - protein, 6 aa, Homo sapiens
QNIHRF

SEQ ID NO: 48 - protein, 9 aa, Homo sapiens
QQYNSYSWT

SEQ ID NO: 49 - protein, 6 aa, Homo sapiens
ALPKQY

SEQ ID NO: 50 - protein, 11 aa, Homo sapiens
HSADISATSWV

SEQ ID NO: 51 - DNA, 376 bp, Homo sapiens
caggtgcagctggtggagtctgggggaggcgtggtccagcctgggaggtccctgagactctcctgtgcagcctctggattcaccttcagtaactatggcatgcactgggtccgccaggctccaggcaaggggctggagtggctggcagttatatcatctgatggaagaattaagttctatgcagactccgtgaagggccgattcaccatgtccagagacagttccaagaacacgctgtatctgcaaatgaacagcctgagagctgaggacacggctgtgtattactgtgcgaaagatcggttccagtttgccagaagctggtacggtgactactttgactactggggccagggaacccaggtcaccgtctcctcag

SEQ ID NO: 52 - protein, 125 aa, Homo sapiens
QVQLVESGGGVVQPGRSLRLSCAASGFTFSNYGMHWVRQAPGKGLEWLAVISSDGRIKFYADSVKGRFTMSRDSSKNTLYLQMNSLRAEDTAVYYCAKDRFQFARSWYGDYFDYWGQGTQVTVSS

SEQ ID NO: 53 - DNA, 337 bp, Homo sapiens
gctgttgcgatgactcagtctccactctccctgcccgtcacccttggacagccggcctccatctcctgcaggtctaatcaaagcctcgtatacagtgatggaaacatctacttgaattggtttcaacagaggccaggccaatctccaatgcgcctaatttatagggtttctaaccgggactctggggtcccagacagattcagcggcagtgggtcaggcactgatttcacactgaaaatcagcagggtggaggctgaagatgttgggatttattactgcatgcaagggacacactggcctccgactttcggcggagggaccaaggtggagatcaaac

SEQ ID NO: 54 - protein, 112 aa, Homo sapiens
AVAMTQSPLSLPVTLGQPASISCRSNQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 55 - DNA, 376 bp, Homo sapiens
caggtcaccttgaaggagtctggtcctgtgctggtgaaacccacagagaccctcacgctgacctgcaccgtctctgtgttctcactcagcaatgctagaatgggtgtgagctggatccgtcagcccccagggaaggccctggagtggcttgcacacattttttcgagtgaccaaaaatcctacagcacatctctgaagagcaggctcaccatctccaaggacacctccaaaagccaggtggtccttaccatgaccaacatggaccctgaggacacaggcacatattactgtgcacgaataaacacggcggcgtatgattatgatagtaccacctttgatatctggggccaagggacaatggtcaccgtctcttcag

SEQ ID NO: 56 - protein, 125 aa, Homo sapiens
QVTLKESGPVLVKPTETLTLTCTVSVFSLSNARMGVSWIRQPPGKALEWLAHIFSSDQKSYSTSLKSRLTISKDTSKSQVVLTMTNMDPEDTGTYYCARINTAAYDYDSTTFDIWGQGTMVTVSS

SEQ ID NO: 57 - DNA, 322 bp, Homo sapiens
gacatccagatgacccagtctccatcctccctgtctgcatctgtaggagacagagtcaccatcacttgccgggcaagtcagaccattagcaactatttaaattggtatcagcagaaaccagggaaagcccctaagctcctgctctatgctgcatccagtttgcaaagtggggtcccatcaaggttcagtggcagtggatctgggacagatttcactctcaccatcagcagtctgcaacttgaagattttgcaacttactactgtcaacagagttacagtacccctcccactttcggcggagggaccaaggtggagatcaaac

SEQ ID NO: 58 - protein, 107 aa, Homo sapiens
DIQMTQSPSSLSASVGDRVTITCRASQTISNYLNWYQQKPGKAPKLLLYAASSLQSGVPSRFSGSGSGTDFTLTISSLQLEDFATYYCQQSYSTPPTFGGGTKVEIK

SEQ ID NO: 59 - DNA, 370 bp, Homo sapiens
caggtgcaactggtggagtctgggggaggcgtggtccagcctgggaggtccctgagactctcctgtgaagcctctggattcaccctcagaaccagtagtctccactgggtccgccaggctccaggcaaggggctggagtgggtggcagttatatcaaatgatggagccactaaattctacgcagacgccgtgaagggccgattcaccatctccagagacaactccaacaacaaaatatatctgcaactgaacggcctgaaacctgaggacacggctgtctattactgtgcgagagaaacacgtcattacagccatggtttgaactggttcgacccctggggccagggaaccctggtcaacgtctcctcag

SEQ ID NO: 60 - protein, 123 aa, Homo sapiens
QVQLVESGGGVVQPGRSLRLSCEASGFTLRTSSLHWVRQAPGKGLEWVAVISNDGATKFYADAVKGRFTISRDNSNNKIYLQLNGLKPEDTAVYYCARETRHYSHGLNWFDPWGQGTLVNVSS

SEQ ID NO: 61 - DNA, 322 bp, Homo sapiens
gaaagagtgatgacgcagtctccagtcaccctgtctgtgtctccaggggaaagagccaccctctcctgcagggccagtcagagtgttagcagcgacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgaccaccttcggccaagggacacgactggacattaaac

SEQ ID NO: 62 - protein, 107 aa, Homo sapiens
ERVMTQSPVTLSVSPGERATLSCRASQSVSSDLAWYQQKPGQAPRLLIYGASTRATGIPARFSGSGSGTEFTLTISSLQSEDFAVYYCQQYNNWPTTFGQGTRLDIK

SEQ ID NO: 63 - DNA, 370 bp, Homo sapiens
caggtgcagctggtggagtctgggggaggcgtggtccagcctgggaggtccctgagactctcctgtgcaggctctggattcaccttcagtagctttggtttgcactgggtccgccaggcgccaggcaagggactggagtggttggcacttatttcagatgagggacgcattaaatactacgcaaactccgtgaagggccgattctttatctccagagacaattccaagaacacgctgtatctgcaaatgaacagcctgagaggtgaggacacggctgtatattactgtgcgagagatgtcaaaggacatattgtggtgatgacttctcttgactactggggccagggagccctggtcaccgtctcctcag

SEQ ID NO: 64 - protein, 123 aa, Homo sapiens
QVQLVESGGGVVQPGRSLRLSCAGSGFTFSSFGLHWVRQAPGKGLEWLALISDEGRIKYYANSVKGRFFISRDNSKNTLYLQMNSLRGEDTAVYYCARDVKGHIVVMTSLDYWGQGALVTVSS

SEQ ID NO: 65 - DNA, 337 bp, Homo sapiens
gatgttgtgatgactcagtctccactctccctgcccgtcacccttggacagccggcctccatctcctgtaggtctagtcaaagcctcgtctccagtgatggagacacctccttgagttggtttcagcagaggccaggccaatctccaaggcgcctaatttatgaggtttctaaccgggactctggggtcccagacagattcagcggcagtgggtcaggcactgatttcacactgaaaatcagcagggtggaggctgaggatgttggggtttattactgcatgcaaggtacacactggcctccgacgttcggccaagggaccaaggtggaaatcaaac

SEQ ID NO: 66 - protein, 112 aa, Homo sapiens
DVVMTQSPLSLPVTLGQPASISCRSSQSLVSSDGDTSLSWFQQRPGQSPRRLIYEVSNRDSGVPDRFSGSGSGTDFTLKISRVEAEDVGVYYCMQGTHWPPTFGQGTKVEIK

SEQ ID NO: 67 - DNA, 379 bp, Homo sapiens
caggtgcttctggtggagtctgggggaggcgtggtccagcctgggatgtccctgagactctcctgtgcagcctctggattcaccttcagttcctatgctatgcactgggtccgccaggctccaggcaaggggctggagtgggtggcagttatctcatatgatggaagcactaaattctacgcagactccgtgaggggccgattccccatctccagagacaattccaagaacacggtgtatctgcaaatgaacagcctgagacctgaggacacggcagtctattactgtgcgacagttagtgtcgaggggtataccagtggctggtatttgggaacccttgacttctggggccagggaaccccggtcaccgtctcctcag

SEQ ID NO: 68 - protein, 126 aa, Homo sapiens
QVLLVESGGGVVQPGMSLRLSCAASGFTFSSYAMHWVRQAPGKGLEWVAVISYDGSTKFYADSVRGRFPISRDNSKNTVYLQMNSLRPEDTAVYYCATVSVEGYTSGWYLGTLDFWGQGTPVTVSS

SEQ ID NO: 69 - DNA, 322 bp, Homo sapiens
gaaagagtgatgacgcagtttccagccaccctgtctgtgtctccaggggaaagagccaccctctcctgcagggccagtcagagtgttagtagcaacttagcctggtaccagcagaaacctggccaggctcccaggctcctcatctatggtgcatccaccagggccattggtgtcccagccaggttcagtggcagtgggtctgggacagagttcactctcaccatcagcagcctgcagtctgaagattttgcagtttattactgtcagcagtataataactggccgggaacttttggccaggggaccaagctggagatcaaac

SEQ ID NO: 70 - protein, 107 aa, Homo sapiens
ERVMTQFPATLSVSPGERATLSCRASQSVSSNLAWYQQKPGQAPRLLIYGASTRAIGVPARFSGSGSGTEFTLTISSLQSEDFAVYYCQQYNNWPGTFGQGTKLEIK

SEQ ID NO: 71 - DNA, 382 bp, Homo sapiens
gaagtgctcctggtggagtctgggggaggcttggtacagcctggcaggtccctgagactctcctgtgtagtctctggattcacctttgattattatgccatgcactgggtccggcaagctccagggaagggcctggagtgggtctcaggtattagttggaatagtgataacacagactatgcggactctgtgaagggccgattcaccatctccagagacaacgccaagaactccctgtatctgcaaatgaacagtctgaaaactgaggacacggccttgtattactgtgcaaaagatattagtctagttttttggagtgttaaccctccccgtaacggaatggacgtctggggccaagggaccacggtcaccgtctcctcag

SEQ ID NO: 72 - protein, 127 aa, Homo sapiens
EVLLVESGGGLVQPGRSLRLSCVVSGFTFDYYAMHWVRQAPGKGLEWVSGISWNSDNTDYADSVKGRFTISRDNAKNSLYLQMNSLKTEDTALYYCAKDISLVFWSVNPPRNGMDVWGQGTTVTVSS

SEQ ID NO: 73 - DNA, 322 bp, Homo sapiens
gacatccagatgacccagtctccatcctccctgtctgcatctgtaggagacagagtcaccatcacttgccgggcaagtcagagcattcgcagctatttaaattggtatcagcagaaaccagggaaagcccctaacctcctgatctatactgcatccagtttgcaaagtggggtcccatcaaggttcagtggcagtggatctgggacagatttcactctcaccatcagcagtctgcaacctgaagattttgcgacttactactgtcaacagagttacagttcccctctcactttcggcggagggaccaaggtggagatcaaac

SEQ ID NO: 74 - protein, 107 aa, Homo sapiens
DIQMTQSPSSLSASVGDRVTITCRASQSIRSYLNWYQQKPGKAPNLLIYTASSLQSGVPSRFSGSGSGTDFTLTISSLQPEDFATYYCQQSYSSPLTFGGGTKVEIK

SEQ ID NO: 75 - DNA, 373 bp, Homo sapiens
ctggtgcaactggtagagtctgggggaggcgtggtccagcctgggaggtccctgagactctcctgtgcaggctctggattcacctttagcagctatggcatgcactgggtccgccagactccaggcaaggggctggagtgggtggcagttatatcgtttgatggaaggaacaaattctacgcagaccccgtgaagggtcgattcaccatctccagagacaattccaagaacacggtgttcttggaattggatagcctgacaactgaggacacggctttttattactgtgcgagagacgacaacttggacagacactggccccttcgactcgggggttactggggccagggaaccctggtcaccgtctcctcag

SEQ ID NO: 76 - protein, 124 aa, Homo sapiens
LVQLVESGGGVVQPGRSLRLSCAGSGFTFSSYGMHWVRQTPGKGLEWVAVISFDGRNKFYADPVKGRFTISRDNSKNTVFLELDSLTTEDTAFYYCARDDNLDRHWPLRLGGYWGQGTLVTVSS

SEQ ID NO: 77 - DNA, 337 bp, Homo sapiens
gaaattgtgatgactcagtctccactctccctgcccgtcacccttggacagccggcctccatctcctgcaggtctagtcaagacctcctatacaatgatggaggcaccgacttgaactggtttcagcagaggccaggccaatctccaaggcgcctaatttacagggtttctaaccgggactctggggtcccagacagattcagcggcagtgggtcaggcagtgatttcacactgaaaatcagcagggtggaggctgaggatgttggaatttattactgcatgcaaggtgcacactggcctccgactttcggccctgggaccaaagtggagatcaaac

SEQ ID NO: 78 - protein, 112 aa, Homo sapiens
EIVMTQSPLSLPVTLGQPASISCRSSQDLLYNDGGTDLNWFQQRPGQSPRRLIYRVSNRDSGVPDRFSGSGSGSDFTLKISRVEAEDVGIYYCMQGAHWPPTFGPGTKVEIK

SEQ ID NO: 79 - DNA, 382 bp, Homo sapiens
caggcgcaactggtggagtctgggggagccttggtccagcctgggaggtccctgagactctcctgtgcagcctctggattcaccttcaggaattatgctatgcactgggtccgccaggctccagccacggggctgcagtggctggcagtcataacatctgatggaaggaataaattctatgcagactccgtgaagggccgattcaccatctccagagaggattccaagaacacgctgtatctgcaaatggatagcctgagaggagaggacacggctgtctactactgcgtgacacagcgtgataatagtcgcgattacttcccccactacttccacgacatggacgtctggggccaagggaccacggtcgccgtctcctcag

SEQ ID NO: 80 - protein, 127 aa, Homo sapiens
QAQLVESGGALVQPGRSLRLSCAASGFTFRNYAMHWVRQAPATGLQWLAVITSDGRNKFYADSVKGRFTISREDSKNTLYLQMDSLRGEDTAVYYCVTQRDNSRDYFPHYFHDMDVWGQGTTVAVSS

SEQ ID NO: 81 - DNA, 337 bp, Homo sapiens
gatgttgtgctgactcagtctccactctccctgcccgtcacccttggacagccggcctccatctcctgcaggtctagtcaaagcctcgtttacagtgatggagacacctacttgaattggtttcagcagaggccaggccaatctccaaggcgcctaatttatcaggtttctaatcgggactctggggtcccagacagatttagcggcagtgggtcaggcactgatttcacactgaaaatcagcagggtggaggctgaggatgttggggtttattactgcatgcaaggttcacactggcctccgacgttcggccaagggaccaaggtggaaatcaaac

SEQ ID NO: 82 - protein, 112 aa, Homo sapiens
DVVLTQSPLSLPVTLGQPASISCRSSQSLVYSDGDTYLNWFQQRPGQSPRRLIYQVSNRDSGVPDRFSGSGSGTDFTLKISRVEAEDVGVYYCMQGSHWPPTFGQGTKVEIK

SEQ ID NO: 83 - DNA, 358 bp, Homo sapiens
gacgtgcagctgttggagtctgggggaggcttggtacagcctggggggtccctgagactctcctgtgcagcctctggattcagctttagcagctatgccatgacctgggtccgccaggctccagggaaggggctggagtgggtcgcaactatgagtgctagtggggatagcacaaacgacgcagactccgtgaagggccggttcaccatctccagagacaattccaagaacacgctgtttctgcaaatgaacagcctcagacccgaggacacggccgtatattactgtgcgtccccccttcggaattatggtgacttgctctactggggccagggaaccctggtcaccgtctcctccg

SEQ ID NO: 84 - protein, 119 aa, Homo sapiens
DVQLLESGGGLVQPGGSLRLSCAASGFSFSSYAMTWVRQAPGKGLEWVATMSASGDSTNDADSVKGRFTISRDNSKNTLFLQMNSLRPEDTAVYYCASPLRNYGDLLYWGQGTLVTVSS

SEQ ID NO: 85 - DNA, 322 bp, Homo sapiens
gacatccagatgacccagtctccttccaccctgtctgcatctgtaggagacagagtcaccatcacttgccgggccagtcagaatattcatcgttttttggcctggtatcagcagaaaccagggaaagcccctaaactcctgatctatacggcgtctagtttagaaagtggggtcccatcaaggttcagcggcagtggatttgggacagaattcactctcaccatcagcagcctgcagcctgatgattttgcaacttattactgccaacaatataatagttactcgtggacgttcggccaagggaccaaggtggaaatcaaac

SEQ ID NO: 86 - protein, 107 aa, Homo sapiens
DIQMTQSPSTLSASVGDRVTITCRASQNIHRFLAWYQQKPGKAPKLLIYTASSLESGVPSRFSGSGFGTEFTLTISSLQPDDFATYYCQQYNSYSWTFGQGTKVEIK

SEQ ID NO: 87 - DNA, 379 bp, Homo sapiens
gaagtgcagttgttggagtctgggggaggcttggttcagccgggggggtccctgagactctcctgtacagcctctggattcacctttgccagctatgccatgacctgggtccgccaggctccaggcaaggggctggagtgggtctcaactattagtggtggtggtggtgacacatactccgcagactccgtgaagggccggttcaccatctccagagacaattccaagagcacgctgtatttgcaaatgaacagcctgagagccgcggacacggccctatattactgtgcgagattggaaagcgcgacgcagcccctcggctactatttctacggtatggacgtctggggccaagggactacggtcaccgtctcctcag

SEQ ID NO: 88 - protein, 126 aa, Homo sapiens
EVQLLESGGGLVQPGGSLRLSCTASGFTFASYAMTWVRQAPGKGLEWVSTISGGGGDTYSADSVKGRFTISRDNSKSTLYLQMNSLRAADTALYYCARLESATQPLGYYFYGMDVWGQGTTVTVSS

SEQ ID NO: 89 - DNA, 328 bp, Homo sapiens
tcctatgagctgacacagccaccctcggtgtcagtgtccccaggacagacggccaggatcacctgctctggagatgcattgccaaagcaatatgctttttggtaccagcagaggccaggccaggcccctgtgttggtgatatctaaagacagtgagaggccctcagggatccctgagcgattctctggctccagctcagggacaacagtcacgttgaccatcagtggagtccaggcagaagacgaggctgactattactgtcattcagcagacatcagtgctacttcttgggttttcggcggagggaccaagctgaccgtcgttagtc

SEQ ID NO: 90 - DNA, 328 bp, Homo sapiens
tcctacgagctgacacagccaccctcggtgtcagtgtccccaggacagacggccaggatcacctgctctggagatgcattgccaaagcaatatgctttttggtaccagcagaggccgggccaggcccctgtgttggtgatatctaaagacagtgagaggccctcagggatccctgagcgattctctggctccagctcagggacaacagtcacgttgaccatcagtggagtccaggcagaagacgaggcagactattactgtcattcagcagacatcagtgctacttcttgggttttcggcggagggaccaagctgaccgtcgttagtc

SEQ ID NO: 91 - protein, 109 aa, Homo sapiens
SYELTQPPSVSVSPGQTARITCSGDALPKQYAFWYQQRPGQAPVLVISKDSERPSGIPERFSGSSSGTTVTLTISGVQAEDEADYYCHSADISATSWVFGGGTKLTVVS

(19) United States
(12) Patent Application Publication
(10) Pub. No.: US 2011/0159001 A1
(43) Pub. Date: Jun. 30, 2011

(54) CROSS-NEUTRALIZING HUMAN MONOCLONAL ANTIBODIES TO SARS-CoV AND METHODS OF USE THEREOF

(75) Inventor: Antonio Lanzavecchia, Bellinzona (CH)

(73) Assignee: INSTITUTE FOR RESEARCH IN BIOMEDICINE, Bellinzona, CA (US)

(21) Appl. No.: 12/863,220

(22) PCT Filed: Jan. 16, 2009
(86) PCT No.: PCT/US09/31299

§ 371 Date: Oct. 29, 2010

Related U.S. Application Data
(60) Provisional application No. 61/021,798, filed on Jan. 17, 2008.

(51) Int. Cl. A61K 39/395; C07K 16/10; C07H 21/00; A61P 31/14

(52) U.S. Cl. 424/147.1; 530/388.3; 530/387.9; 536/23.53

(57) ABSTRACT

This invention relates generally to human monoclonal antibodies against SARS-CoV, epitopes bound by the bodies as well as to methods for use thereof.

This application claims priority to U.S. provisional application No. 61/021,798, filed Jan. 17, 2008, the disclosure of which, along with all documents cited therein, is incorporated by reference in its entirety.

### BACKGROUND

This invention relates generally to human monoclonal antibodies against SARS-CoV s as well as to methods for use thereof.

In 2002-2003 a novel Coronavirus caused an outbreak of Severe Acute Respiratory Syndrome (SARS-CoV) which infected over 8000 people, and was associated with ˜10% fatality rate (4, 21). In addition several laboratory acquired cases of SARS-CoV infection were reported in 2003 and 2004 including community spread, highlighting a need for therapeutics (27, 33). Old age (>60-years-old) was significantly associated with increased SARS-related deaths due to rapidly progressive respiratory compromise (acute respiratory distress syndrome [ARDS]) (4, 28, 42).

SARS-CoV is a zoonotic virus most likely originating from Chinese horseshoe bats, amplified in palm civets and raccoon dogs in the live animal markets, and subsequently transmitted into human populations (17). The 2003-2004 epidemic has been divided into zoonotic, early, middle and late phases based on molecular epidemiological studies (6). Comparative analysis of the SARS-CoV genomes from both human and zoonotic isolates throughout the different phases of the epidemic showed a high rate of evolution in the viral attachment protein, the spike (S) glycoprotein, with 23 amino acid changes evolving over the course of the epidemic (39).

Several studies have shown that the SARS-CoV spike glycoprotein binds to the receptor Angiotensin 1 converting enzyme 2 (ACE-2), mediating viral entry (24, 54). A total of 18 amino acids in ACE2 have been identified that are in contact with 14 residues in the receptor-binding domain (RBD) of SARS-CoV (23). Two of these amino acids, 479 and 487, have been shown to be critical in binding of the RBD to human ACE2 and linked to cross species transmission into humans during the epidemic. Not surprisingly, the spike (S) glycoprotein has also been identified as a major component of protective immunity and is highly immunogenic containing at least three domains that are targeted by neutralizing antibodies (11, 14, 22). The exact number of neutralizing epitopes is unknown as is the effect of the sequence variation in these regions on neutralization between the different S glycoprotein isolated during the SARS-CoV epidemic.

Both human and murine monoclonal antibodies (mAbs) have been developed against three late phase SARS-CoV strains, including Urbani, Tor-2 and HKU-39849, and neutralizing activity has been described in vitro (48-50). The recent development of a method to isolate a large number of monoclonal antibodies from SARS patient provides the reagents needed to characterize the homologous and heterologous neutralizing responses after natural SARS-CoV infection (49). Although studies using pseudotyped lentiviruses and recombinant SARS-CoV RBD protein have shown some cross-neutralizing or cross-reactive activity (13, 26, 45, 58, 60), the neutralizing activity of these mAbs has not been tested against actual heterologous SARS-CoV strains from the middle, early or zoonotic phases of the epidemic, or in lethal models of disease. This is potentially problematic as the absence of human cases over the past two years suggests that future epidemics will likely result from zoonotic transmission. Consequently, antibodies that provide robust cross neutralization activity are essential to interrupt zoonotic transmission and contain future epidemics (3, 38).

Passive immunization studies with selected mAbs in mice, ferrets and hamsters, have demonstrated that some neutralizing antibodies can successfully prevent or limit infection (37, 45, 47, 49). While prophylactic treatment can result in complete protection from SARS-CoV infection in rodents, post-infection treatment is usually less robust but significantly reduces viral titers in the lung (37). To date, all previous studies were performed in young animals, which allow for virus replication in the absence of notable clinical symptoms and disease (39, 44). So, based on art to date, it is not simply a given that antibodies will prevent clinical disease or provide measurable levels of protection against homologous or heterologous lethal challenge, especially in more vulnerable senescent populations.

Passive protection of senescent populations has also been poorly studied, yet aged populations are most vulnerable to severe and fatal SARS-CoV infection (4, 28, 42). In the aged BALB/c mouse model, passive transfer of hyper immune SARS-CoV antiserum from mice prevented infection with the homologous late phase Urbani strain (53). The use of human mAb for prevention or treatment of lethal heterologous SARS-CoV infection in aged populations, however, has not been studied in detail. In addition, a recently reported vaccine failure in aged populations makes passive immunization an attractive alternative (8).

In light of the above, effective prophylaxis and therapies are urgently needed in the event that there is reemergence of the highly contagious and often lethal severe acute respiratory syndrome (SARS) Coronavirus (SARS-CoV) infection. Currently, prevention of SARS has largely relied on improved awareness, surveillance, and institution of local, regional and international public-health-care measures (see Stadler et al, Nat Rev Microbiol 1:209-18 (2003)). Significant efforts in the area of SARS vaccine research have been initiated and several recent reports have documented that transfer of immune serum from mice with prior SARS-CoV infection, or from mice vaccinated with a DNA plasmid encoding SARS S protein or a vaccinia virus expressing the S protein, can prevent virus replication in the lungs and upper respiratory tract (see Bisht et al, Proc. Natl Acad Sci USA 101:6641-46 (2004); Subbarao et al, J Virol 78:3572-77 (2004); Yang et al, Nature 428:561-64 (2004)). In addition, in SARS-CoV infection of humans, decreasing virus titers from nasopharyngeal aspirates, serum, urine and stool have been observed to be coincident with the development of neutralizing antibodies (see Li et al, N Engl Med 349:508-09 (2003); Peiris et al, Lancet 361:1767-72 (2003)). Treatment of SARS with convalescent plasma has been reported (see Burnouf et al, Hong Kong Med. J. 9:309-10 (2003); Wong et al, Hong Kong Med. J. 9:199-201 (2003)).

These studies support the importance of humoral immunity in protection against SARS-CoV and suggest that specific and effective human monoclonal antibodies (mAbs) should be developed to provide a prophylaxis and early treatment against SARS in the event that episodic or even widespread reemergence into the human population occurs. Ideally, effective human monoclonal antibodies that cross neutralize multiple strains would confer the best protection from world health perspective.

### SUMMARY OF INVENTION

The invention is based, in part, on the discovery of antibodies that cross neutralize different strains of SARS-CoV as well as novel epitopes to which the antibodies of the invention bind. Accordingly, in one embodiment, the invention comprises a monoclonal antibody that cross neutralizes at least three strains of SARS-CoV.

In another embodiment, the invention comprises an epitope that binds to an antibody of the invention. Exemplary epitopes of the invention include, but are not limited to, an epitope comprising amino acids from SARS-CoV spike protein.

In yet another embodiment, the invention comprises an immunogenic composition comprising amino acids from SARS CoV spike protein and optionally, a pharmaceutically acceptable carrier.

In yet another embodiment, the invention comprises a method of preventing a disease or disorder caused by a coronavirus. The method comprises administering to a person at risk of suffering from the disease or disorder a therapeutically effective amount of one or more monoclonal antibodies of the invention.

Other features and advantages of the invention will be apparent from the following detailed description and claims.

### BRIEF DESCRIPTION OF THE DRAWINGS

FIG. 1. Mapping of neutralizing epitopes on the SARS-CoV S glycoprotein, recognized by human mAbs through phylogenetic analysis and cross-competition studies. (A) Phylogenetic analysis of the amino acid changes in the SARS-CoV S glycoprotein of zoonotic and human epidemic isolates. The graphic representation of the SARS-CoV S glycoprotein shows the locations of the variant amino acids in the receptor binding domain (RBD), putative fusion peptide (FP) and the heptad-repeat 2 (HR2). (B) Cross-competition of mAbs binding to the SARS-CoV S glycoprotein. Shown is the inhibition of binding of 3 biotinylated mAbs: S109.8 (black bar), S227.14 (grey bar) and S230.15 (white bar) to the recombinant SARS-CoV S glycoprotein by a panel of 23 mAbs belonging to groups I through VI. Values represent the percentage (%) of inhibition of the biotinylated mAb at 0.1 μg/ml by unlabeled competing mAb at saturating concentrations (5 μg/ml). Error bars represent the standard deviation from triplicates.

FIG. 2. Location and effects of neutralization escape variant mutations on the structure of the SARS-CoV RBD. (A) The S109.8 escape variant mutations T332I and K333N and (B) S230.15 escape variant mutation L443R were mapped onto the structure of the SARS-CoV RBD. (C) In addition the locations of all the important amino acid residues associated with the cross neutralizing mAbs were highlighted in the SARS-CoV RBD. Amino acid residues associated with S109.8, S227.14, and S230.15 are indicated.

FIG. 3. Prophylactic treatment of lethal SARS-CoV infection in 12-month-old BALB/c mice with 25 pg of cross neutralizing mAbs. Body weights of mice infected with icUrbani (A), icGZ02 (B) and icHC/SZ/61/03 (C) were measured daily after passive transfer of 25 μg of mAbs S109.8 (+), S227.14 (∘), S230.15 (×) and D2.2 (□, a control mAb of irrelevant specificity). Lung tissues were harvested from infected mice on day 2 (D) and day 5 (E) post infection and assayed for infectious virus. Error bar represent standard deviations (n=3).

FIG. 4. Prophylactic treatment of lethal SARS-CoV infection in 12-month-old BALB/c mice with 250 μg of cross neutralizing mAbs. Body weights of mice infected with icUrbani (A), icGZ02 (B) and icHC/SZ/61/03 (C) were measured daily after passive transfer of mAbs S109.8 (+), S227.14 (∘), S230.15 (×) and D2.2 (□) all at 250 μg/mouse, given alone or as a 1:1:1 cocktail (Δ). Lung tissues were harvested from infected mice on day 2 (D) and day 5 (E) post infection and assayed for infectious virus. Error bar represent standard deviations (n=3).

FIG. 5. Prophylactic treatment of lethal SARS-CoV infection in 10-week-old BALB/c mice with 25 μg of cross neutralizing mAbs. Body weights of mice infected with MA15 (A) were measured daily after passive transfer of 25 μg of mAbs S109.8 (+), S227.14 (∘), S230.15 (×) and D2.2 (□). Lung tissues of mice infected with MA15 or icHC/SZ/61/03 were harvested on day 2 (B) and day 4 (C) post infection and assayed for infectious virus. Error bar represent standard deviations (n=3). “*” indicates that only one animal out of 3 had detectable virus titers.

FIG. 6. Post infection treatment of 12-month-old BALB/c mice infected with SARS-CoV. Body weights of mice infected with GZ02 (A) were measured daily after passive transfer of 250 μg of mAbs S230.15 at day −1 (+), day 0 (∘), day 1 (×), day 2 (□) and day 3 (Δ) post infection. Lung titers of mice infected with GZ02 (B) were harvested on day 2 and day 4 post infection and assayed for infectious virus. Error bar represent standard deviations (n=5). “*” indicates that only one animal out of 5 had detectable virus titers.

FIG. 7. Light photographs of preterminal (PB) bronchioles in the lungs of 12-month-old BALB/c mice that received 250 μg of a human mAb prior to SARS-CoV infection and sacrificed 5 days postinoculation. Virus induced peribronchiolar inflammation (solid arrows) is evident in mice treated with the control mAb D2.2 and infected with icUrbani (A), icGZ02 (C) and icHC/SZ/61/03 (E). Numerous hyaline membranes (dotted arrows) are present in the alveolar airspaces of mice treated with the control mAb. No inflammation or hylaline membrane formation can be observed in mice treated with 250 μg of mAb S230.15 and subsequently infected with icUrbani (B), icGZO2 (D) and icHC/SZ/61/03 (F). AL, alveoli, AD, alveolar ducts, BV, blood vessels. Tissues were stained with hematoxylin and eosin. 100× magnification.

FIG. 8. Light photographs of preterminal (PB) and terminal (TB) bronchioles in the lungs of 12-month-old BALB/c mice that received 250 μg of a human mAb post-infection with SARS-CoV and sacrificed 5 days postinoculation. No inflammation or hylaline membrane formation can be observed in mice treated with 250 μg of mAb S230.15 on day 0 of infection with icGZO2 (A). Increasing virus induced peribronchiolar inflammation (solid arrows) is evident in mice treated with 250 μg of mAb S230.15 at days 1 (B), 2 (C) or 3 (D) post infection. AL, alveoli, AD, alveolar ducts, BV, blood vessels. Tissues were stained with hematoxylin and eosin. 100× magnification.

### DETAILED DESCRIPTION OF THE INVENTION

The invention is based on the discovery of antibodies that cross neutralize different strains of SARS. Accordingly, in one aspect, the invention comprises a monoclonal antibody that cross neutralizes at least three strains of SARS-CoV.

Several lethal SARS-CoV challenge models have been developed in BALB/c mice that recapitulated the age related clinical signs, weight loss exceeding 20% as well as severe lung pathology, by using recombinant SARS-CoV bearing the S glycoprotein of early human and zoonotic strains (39). A second pathogenic model for young mice was also developed by serial passage of the Urbani isolate in BALB/c mice, resulting in MA15 which replicates to high titers in the lung, causes clinical disease, weight loss exceeding 20% and severe alveolitis (35). A panel of isogenic SARS-CoV bearing human and zoonotic S glycoproteins was used to subdivide human mAbs into six distinct neutralization profiles. Four neutralizing mAbs were identified that neutralize all zoonotic and human SARS-CoV strains tested, and demonstrate that three of these mAbs engage unique epitopes in the S glycoprotein providing for the development of a broad spectrum therapeutic that protects young and senescent mice from lethal homologous and heterologous challenge. Any of these, or a cocktail of more than one of these, mAbs would provide robust protection from lethal SARS-CoV infection in humans. In one aspect, the present invention concerns these novel mAbs, therapeutic compositions comprising the antibodies, and methods of their production and use in the treatment of SARS.

Definitions of terms and further description of novel antibodies, compositions comprising them, and methods practicable with the antibodies provided herein are given below.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

### Antibodies

The invention provides monoclonal or recombinant monoclonal antibodies (both referred as mAbs) having particularly high potency in neutralizing SARS-CoV. The invention also provides antibodies that cross neutralize multiple, e.g., at least three strains of SARS-CoV. The invention also provides fragments of these recombinant or monoclonal antibodies, particularly fragments that retain the antigen-binding activity of the antibodies, for example which retain at least one complementarity determining region (CDR) specific for SARS-CoV proteins.

As used herein, the term “antibody” refers to immunoglobulin molecules and immunologically active portions of immunoglobulin (Ig) molecules, i.e., molecules that contain an antigen binding site that specifically binds (immunoreacts with) an antigen. In general, antibody molecules obtained from humans relate to any of the classes IgG, IgM, IgA, IgE and IgD, which differ from one another by the nature of the heavy chain (“H,” see below) present in the molecule. Certain classes have subclasses as well, such as IgG₁, IgG₂, and others. Furthermore, in humans, the light chain (“L”) may be a kappa chain or a lambda chain.

The terms “fragment,” and “antibody fragment” are used interchangeably herein to refer to any fragment of an antibody of the invention that retains the antigen-binding activity of the antibodies. Exemplary antibody fragments include, but are not limited to, Fab, Fab′, F(ab′)2 and Fv fragments.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

By “specifically binds” or “immunoreacts with” is meant that the antibody reacts with one or more antigenic determinants of the desired antigen and does not react with other polypeptides. Antibodies include, but are not limited to, polyclonal, monoclonal, chimeric, dAb (domain antibody), single chain, F_{ab}, Fab_{ab′} and F_{(ab′)2} fragments, scFvs, and F_{ab} expression libraries.

A single chain Fv (“scFv”) polypeptide molecule is a covalently linked V_{H}::V_{L} heterodimer, which can be expressed from a gene fusion including V_{H}- and V_{L}-encoding genes linked by a peptide-encoding linker. (See Huston et al. (1988) Proc Nat Acad Sci USA 85(16):5879-5883). A number of methods have been described to discern chemical structures for converting the naturally aggregated, but chemically separated, light and heavy polypeptide chains from an antibody V region into an scFv molecule, which will fold into a three dimensional structure substantially similar to the structure of an antigen-binding site. See, e.g., U.S. Pat. Nos. 5,091,513; 5,132,405; and 4,946,778. Very large naïve human scFv libraries have been and can be created to offer a large source of rearranged antibody genes against a wide array of target molecules. Smaller libraries can be constructed from individuals with infectious diseases in order to isolate disease-specific antibodies. (See Barbas et al, Proc. Natl. Acad. Sci. USA 89:9339-43 (1992); Zebedee et al, Proc. Natl. Acad. Sci. USA 89:3175-79 (1992)).

As used herein, the term “epitope” includes any determinant capable of specific binding to an immunoglobulin, a scFv, or a T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody as provided herein is said to specifically bind to a SARS-CoV epitope when the equilibrium binding constant (K_{d}) is 1 μM, preferably 100 nM, more preferably 10 nM, and most preferably 100 pM to about 1 pM, as measured by assays such as radioligand binding assays or similar assays known to those skilled in the art.

The term “monoclonal antibody” or “mAb” or “monoclonal antibody composition”, as used herein, refers to a population of antibody molecules that contains only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it. The antibodies of the invention may be monoclonal, for example, human monoclonal antibodies, or recombinant antibodies. The invention also provides fragments of the antibodies of the invention, particularly fragments that retain the antigen-binding activity of the antibodies.

A “neutralizing antibody” is one that can neutralize the ability of that pathogen to initiate and/or perpetuate an infection in a host. The invention provides a neutralizing monoclonal human antibody, wherein the antibody recognizes an antigen from human SARS-CoV.

The antibodies of the invention are able to cross neutralize different strains of SARS-CoV. In one embodiment, the antibodies of the invention are capable of cross neutralizing at least three strains of SARS-CoV. In another embodiment, the antibodies are able to cross neutralize at least four strains of SARS-CoV. In yet another embodiment, the antibodies are capable of neutralizing at least 4 or 5 strains of SARS-CoV. The antibodies of the invention are capable of neutralizing of human and zoonotic SARS-CoV strains.

Several different strains of SARS-CoV are known to one of skill in the art. Exemplary SARS-CoV strains include, but are not limited to, Urbani, CUHK-W, GZ02, HC/SZ/61/03, and A031G.

In one embodiment, the monoclonal antibodies of the invention bind an epitope present on a SARS-CoV spike protein. As used herein, the terms “spike protein,” “SARS-CoV spike protein” and “SARS-CoV S glycoprotein” are used interchangeably. These terms as well as the specific aminoacid positions of the SARS-CoV spike protein refer to the protein and the aminoacid sequence of the epidemic strain virus Urbani (GenBank accession number is AAP13441).

Exemplary epitopes bound by the antibodies of the invention include, but are not limited to, those that comprise an amino acid at positions 332, 333, 390, 436, 443, or 487 of the SARS-CoV Spike protein. In one embodiment, the antibodies of the invention bind to an epitope that comprises at least 2 amino acids at, for example, positions 332, 333, 390, 436, 443, or 487 of the SARS-CoV Spike protein. An antibody of the invention may for example, bind amino acids at positions 332 and 333, or amino acids at positions 443 and 487 of the SARS-CoV Spike protein. In another embodiment, the antibodies of the invention bind to an epitope that comprises at least 3 amino acids at, for example, positions 332, 333, 390, 436, 443, or 487 of the SARS-CoV Spike protein. An antibody of the invention may for example, bind amino acids at positions 436, 443 and 487.

It is understood by one skilled in the art that amino acid changes in the target antigen can decrease the efficacy of the neutralizing antibody. For instance, selective pressure by neutralizing antibodies can result in the isolation of escape mutants of viruses. In one embodiment, the neutralizing antibody to SARS-CoV is directed toward the spike (S) protein. In another embodiment, amino acid changes in the S protein decrease the efficacy of the neutralizing antibody by about ten-fold.

In one embodiment, the neutralization ability of a mAb of the invention is decreased by a mutation in the SARS CoV spike protein. Exemplary amino acid changes in the SARS-CoV spike protein that affect neutralization of the SARS-CoV by an antibody of the invention include, but are not limited to, those at amino acid positions 332, 333, 390, 436, 443 or 487. Mutations at these amino acid positions may decrease neutralization ability of a mAb of the invention. In one embodiment, the mutation that results in decreased neutralization ability is selected from the group consisting of L443R, T332I, K333N, K390Q, K390E, Y436H, and T487S.

In general, the antibodies of the invention have high affinity, for example an affinity of 10⁻⁶M or less (i.e., 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, or 10⁻¹¹M or less) for the SARS-CoV Spike protein.

In this specification, by “high potency in neutralizing SARS-CoV” is meant that an antibody molecule of the invention neutralizes SARS-CoV in a standard assay at a concentration much lower than antibodies known in the art.

In one embodiment, the antibody molecule provided herein can neutralize at a concentration of 5.6 μg/ml or lower (i.e., at 5, 4.5, 4, 3.5, 3, 2.5, 2, 1.5, 1, 0.5 μg/ml or lower). In another embodiment, the antibody molecule of the invention can neutralize at a concentration of 3 μg/ml or lower (i.e., at 2.5, 2, 1.5, 1, 0.8, 0.6, 0.4, 0.2 μg/ml or lower). In yet another embodiment, the antibody can neutralize at a concentration of 1 μg/ml or lower (i.e., at 0.8, 0.6, 0.4, 0.3, 0.25, 0.2, 0.15, 0.1 μg/ml or lower). In yet another embodiment, the antibody can neutralize at a concentration of 0.4 μg/ml or lower (i.e., at 0.3, 0.25, 0.2, 0.16, 0.12, 0.08, 0.05, 0.04, 0.03, 0.02, 0.01 μg/ml or lower). In yet another embodiment, the antibody can neutralize at a concentration of 0.16 μg/ml or lower (i.e. 0.15, 0.125, 0.1, 0.075, 0.05, 0.025, 0.02, 0.016, 0.015, 0.0125, 0.01, 0.0075, 0.005, 0.004 μg/ml or lower). In a further embodiment, the antibody can neutralize at a concentration of 0.016 μg/ml or lower (10⁻⁹ M or lower, 10⁻¹⁰ M or lower, 10⁻¹¹M or lower, 10⁻¹²M or lower, 10⁻¹³M or lower). This means that only very low concentrations of antibody are required for 50% neutralization of a clinical isolate of SARS-CoV in vitro compared to the concentration required for neutralization of the same titer of SARS-CoV. Potency can be measured using a standard neutralization assay as described in the art.

In certain embodiments, provided herein is a mAb referred to as S227.14, S230.15, or S109.8. Antibody S227.14 consists of a heavy chain having the amino acid sequence recited in SEQ ID NO: 94 and a light chain having the amino acid sequence recited in SEQ ID NO: 96. Antibody S230.15 consists of a heavy chain having the amino acid sequence recited in SEQ ID NO: 90 and a light chain having the amino acid sequence recited in SEQ ID NO: 92. Antibody S109.8 consists of a heavy chain having the amino acid sequence recited in SEQ ID NO: 98 and a light chain having the amino acid sequence recited in SEQ ID NO: 101.

The CDRs of the antibody heavy chains are referred to as CDRH1, CDRH2 and CDRH3, respectively. Similarly, the CDRs of the antibody light chains are referred to as CDRL1, CDRL2 and CDRL3, respectively. The positions of the CDR amino acids are defined according to the IMGT numbering system as: CDR1—IMGT positions 27 to 38, CDR2—IMGT positions 56 to 65 and CDR3—IMGT positions 105 to 117.

The sequences of the CDRs of these antibodies are identified by sequence identification number in Table 1.

TABLE 1

| | S227.14 | S230.15 | S109.8 |
|---|---|---|---|
| CDRH1 | SEQ ID NO: 25 | SEQ ID NO: 22 | SEQ ID NO: 28 |
| CDRH2 | SEQ ID NO: 26 | SEQ ID NO: 23 | SEQ ID NO: 29 |
| CDRH3 | SEQ ID NO: 27 | SEQ ID NO: 24 | SEQ ID NO: 30 |
| CDRL1 | SEQ ID NO: 55 | SEQ ID NO: 52 | SEQ ID NO: 58 |
| CDRL2 | SEQ ID NO: 56 | SEQ ID NO: 53 | SEQ ID NO: 59 |
| CDRL3 | SEQ ID NO: 57 | SEQ ID NO: 54 | SEQ ID NO: 60 |

Also provided is an antibody comprising a heavy chain comprising one or more (i.e. one, two or all three) heavy chain CDRs from S227.14, S230.15, or S109.8 (SEQ ID NOs: 25-27, 22-24, or 28-30).

In certain embodiments, an antibody as provided herein comprises a heavy chain comprising (i) SEQ ID NO: 25 for CDRH1, SEQ ID NO: 26 for CDRH2 and SEQ ID NO: 27 for CDRH3, or (ii) SEQ ID NO: 22 for CDRH1, SEQ ID NO: 23 for CDRH2 and SEQ ID NO: 24 for CDRH3, or (iii) SEQ ID NO: 28 for CDRH1, SEQ ID NO: 29 for CDRH2 and SEQ ID NO: 30 for CDRH3.

Also provided is an antibody comprising a light chain comprising one or more (i.e. one, two or all three) light chain CDRs from S227.14, S230.15, or S109.8 (SEQ ID NOs: 55-57, 52-54, or 58-60).

In certain embodiments, an antibody as provided herein comprises a light chain comprising (i) SEQ ID NO: 55 for CDRL1, SEQ ID NO: 56 for CDRL2 and SEQ ID NO: 57 for CDRL3, or (ii) SEQ ID NO: 52 for CDRL1, SEQ ID NO: 53 for CDRL2 and SEQ ID NO: 54 for CDRL3, or (iii) SEQ ID NO: 58 for CDRL1, SEQ ID NO: 59 for CDRL2 and SEQ ID NO: 30 for CDRL3.

In certain embodiments, an antibody as provided herein comprises a heavy chain having the sequence recited in any one of SEQ ID NOs: 94, 90 and 98. In further embodiments an antibody according to the invention comprises a light chain having the sequence recited in any one of SEQ ID NOs: 96, 92 and 101.

Hybrid antibody molecules may also exist that comprise one or more CDRs from different antibodies as disclosed herein. For example, a hybrid antibody may comprise one or more CDRs from S227.14 and one or more CDRs from S230.15. Alternatively, a hybrid antibody may comprise one or more CDRS from S227.14 and one or more CDRs from S109.8. Alternatively, a hybrid antibody may comprise one or more CDRs from S230.15 and one or more CDRs from S109.8. In certain embodiments, such hybrid antibodies comprise three CDRs from different antibodies as disclosed herein. Thus, in certain embodiments, such hybrid antibodies comprise i) the three light chain CDRs from S227.14 and the three heavy chain CDRs from S230.15, or ii) the three heavy chain CDRs from S227.14 and the three light chain CDRs from S230.15. In an alternative, such hybrids may comprise i) the three light chain CDRs from S227.14 and the three heavy chain CDRs from S109.8, or ii) the three heavy chain CDRs from S227.14 and the three light chain CDRs from S109.8. In another alternative, such hybrids may comprise i) the three light chain CDRs from S230.15 and the three heavy chain CDRs from S109.8, or ii) the three heavy chain CDRs from S230.15 and the three light chain CDRs from S109.8.

Also provided herein are nucleic acid sequences encoding part or all of the light and heavy chains and CDRs provided herein. For example, nucleic acid sequences provided herein include SEQ ID NO: 93 (encoding the S227.14 heavy chain variable region), SEQ ID NO: 95 (encoding the S227.14 light chain variable region), SEQ ID NO: 89 (encoding the S230.15 heavy chain variable region), SEQ ID NO: 91 (encoding the S230.15 light chain variable region), SEQ ID NO: 97 (encoding the S109.8 heavy chain variable region) and SEQ ID NO: 99 and SEQ ID NO: 100 (encoding the S109.8 light chain variable region). Also provided are nucleic acid sequences encoding the various CDRs. Due to the redundancy of the genetic code, variants of these sequences will exist that encode the same amino acid sequences, such as, for example, SEQ ID NOs: 99 and 100 (encoding the S109.8 light chain variable region).

Variant antibodies are also included within the scope of the invention. Thus, variants of the sequences recited in the application are also included within the scope of the invention. Such variants may arise due to the degeneracy of the genetic code, as mentioned above. Alternatively, natural variants may be produced due to errors in transcription or translation. Further variants of the antibody sequences having improved affinity may be obtained using methods known in the art and are included within the scope of the invention. For example, amino acid substitutions may be used to obtain antibodies with further improved affinity. Alternatively, codon optimization of the nucleotide sequence may be used to improve the efficiency of translation in expression systems for the production of the antibody.

In further embodiments, such variant antibody sequences will share 70% or more (i.e. 80, 85, 90, 95, 97, 98, 99% or more) sequence identity with the sequences recited in the application. In further embodiments such sequence identity is calculated with regard to the full length of the reference sequence (i.e. the sequence recited in the application). In further embodiments, percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

Further included within the scope of the invention are vectors, for example expression vectors, comprising a nucleic acid sequence according to the invention. Cells transformed with such vectors are also included within the scope of the invention.

The invention also relates to monoclonal antibodies that bind to an epitope which is bound by the monoclonal antibody S227.14, S230.15, S109.8. An epitope comprises at least 3, 4, 5, 6, 7, 8, 9, or 10 amino acids of SARS CoV S protein. Amino acid positions important for binding and/or neutralization include, but are not limited to, amino acids at positions 332, 333, 390, 436, 443, or 487 of the SARS CoV S protein.

Antibodies as provided herein are preferably provided in purified form. Typically, the antibody will be present in a composition that is substantially free of other polypeptides e.g. where less than 90% (by weight), usually less than 60% and more usually less than 50% of the composition is made up of other polypeptides.

Antibodies as provided herein may be immunogenic in non-human (or heterologous) hosts e.g. in mice. In particular, the antibodies may have an idiotope that is immunogenic in non-human hosts, but not in a human host. Antibodies as provided herein for human use include those that cannot be obtained from hosts such as mice, goats, rabbits, rats, non-primate mammals, etc. and cannot be obtained by humanization or from xeno-mice.

Antibodies as provided herein can be of any isotype (e.g. IgA, IgG, IgM i.e. an α, γor μ heavy chain), but will generally be IgG. Within the IgG isotype, antibodies may be IgG1, IgG2, IgG3 or IgG4 subclass. Antibodies as provided herein may have a κ or a λ light chain.

A SARS-CoV protein, e.g., S1 (spike 1), S2 (spike 2) or M (membrane), or a derivative, fragment, analog, homolog or ortholog thereof, may be utilized as an immunogen in the generation of antibodies that immunospecifically bind these protein components. Those skilled in the art will recognize that it is possible to determine, without undue experimentation, if a human monoclonal antibody has the same specificity as another human monoclonal antibody by ascertaining whether the former prevents the latter from binding to the S1 region of SARS-CoV. If the human monoclonal antibody being tested competes with the human monoclonal antibody as provided herein, as shown by a decrease in binding by the human monoclonal antibody as provided herein, then it is likely that the two monoclonal antibodies bind to the same, or to a closely related, epitope.

Another way to determine whether a human monoclonal antibody has the specificity of a human monoclonal antibody as provided herein is to pre-incubate the human monoclonal antibody with the SARS-CoV S1 protein, with which it is normally reactive, and then add the human monoclonal antibody being tested to determine if the human monoclonal antibody being tested is inhibited in its ability to bind the S1 region. If the human monoclonal antibody being tested is inhibited then, in all likelihood, it has the same, or functionally equivalent, epitope specificity as the monoclonal antibody as provided herein. Screening of human monoclonal antibodies can be also carried out by utilizing SARS-CoV and determining whether the test monoclonal antibody is able to neutralize SARS-CoV.

Monoclonal and recombinant antibodies are also useful in identification and purification of the individual polypeptides or other antigens against which they are directed. The antibodies provided herein have additional utility in that they may be employed as reagents in immunoassays, radioimmunoassays (RIA) or enzyme-linked immunosorbent assays (ELISA). In these applications, the antibodies can be labeled with an analytically-detectable reagent such as a radioisotope, a fluorescent molecule or an enzyme. The antibodies may also be used for the molecular identification and characterization (epitope mapping) of antigens.

These antibodies can be used as prophylactic or therapeutic agents upon appropriate formulation, or as a diagnostic tool.

In another aspect, the invention comprises an epitope that binds to an antibody of the invention. Exemplary epitopes of the invention include, but are not limited to, those comprising amino acids from SARS-CoV spike protein. In one embodiment, an epitope of the invention comprises an amino acid, or at least 2 amino acids, or at least 3 amino acids at positions 332, 333, 390, 436, 443, or 487 of the SARS-CoV Spike protein. The epitope may include, but is not limited to, amino acids at positions 332 and 333, positions 443 and 487, or positions 436, 443 and 487.

### General Methods of Antibody Production

Various procedures known within the art may be used for the production of polyclonal or monoclonal antibodies directed against a protein as provided herein, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, N.Y., incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103) Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. (See Kozbor, J. Immunol., 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. The binding specificity of monoclonal antibodies produced by the hybridoma cells may then be determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies as provided herein can be readily isolated and sequenced using conventional procedures (e.g., by using oligonucleotide probes that are capable of binding specifically to genes encoding the heavy and light chains of human antibodies). Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA can also be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody, or can be substituted for the variable domains of one antigen-combining site of an antibody to create a chimeric bivalent antibody.

Fully human antibodies are antibody molecules in which the entire sequence of both the light chain and the heavy chain, including the CDRs, arise from human genes. Such antibodies are termed “human antibodies,” or “fully human antibodies” herein. Human monoclonal antibodies can be prepared by using trioma technique; the human B-cell hybridoma technique (see Kozbor, et al, 1983 Immunol Today 4: 72); and the Epstein Barr Virus (EBV) transformation technique to produce human monoclonal antibodies (see Cole, et al, 1985 In: Monoclonal Antibodies and Cancer Therapy, Alan R. Liss, Inc., pp. 77-96). Human monoclonal antibodies may be utilized and may be produced by using human hybridomas (see Cote, et al, 1983. Proc Natl Acad Sci USA 80: 2026-2030) or by transforming human B-cells with EBV in vitro (see Cole, et al, citation supra).

In addition, human antibodies can also be produced using additional techniques, including phage display libraries. (See Hoogenboom and Winter, J. Mol. Biol., 227:381 (1991); Marks et al, J. Mol. Biol., 222:581(1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al, Bio/Technology 10, 779-783 (1992); Lonberg et al, Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic non-human animals which are modified so as to produce fully human antibodies rather than the animal's endogenous antibodies in response to challenge by an antigen. (See PCT publication W094/02602). The endogenous genes encoding the heavy and light immunoglobulin chains in the non-human host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a non-human host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, methods for identifying a clinically relevant epitope on an immunogen, and a correlative method for selecting an antibody that binds immunospecifically to the relevant epitope with high affinity, are disclosed in PCT publication WO 99/53049.

The antibody can be expressed by a vector containing a DNA segment encoding the single chain antibody described above.

These can include vectors, liposomes, naked DNA, adjuvant-assisted DNA, gene gun, catheters, etc. Vectors include chemical conjugates such as described in WO 93/64701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpes virus vectors such as a herpes simplex I virus (HSV) vector (see Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87:1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G. et al, Nat. Genet. 8:148 (1994).

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icy) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of SARS-CoV in a sample. The antibody can also be used to try to bind to and disrupt SARS-CoV Interaction with the SARS-CoV receptor ACE2.

Techniques can be adapted for the production of single-chain antibodies specific to an antigenic protein (see e.g., U.S. Pat. No. 4,946,778). In addition, methods can be adapted for the construction of F_{ab} expression libraries (see e.g., Huse, et al, 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Heteroconjugate antibodies are also within the scope of the present invention. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercapto-butyrimidate and those disclosed, for example, in U.S. Pat. No. 4,676,980.

It can be desirable to modify the antibodies as provided herein with respect to effector function, so as to enhance, e.g., the effectiveness of the antibody in treating SARS. For example, cysteine residue(s) can be introduced into the F_{c} region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176: 1191-1195 (1992) and Shopes, J. Immunol., 148: 2918-2922 (1992)). Alternatively, an antibody can be engineered that has dual F_{c} regions and can thereby have enhanced complement lysis and ADCC capabilities. (See Stevenson et al, Anti-Cancer Drug Design, 3: 219-230 (1989)).

### Conjugate Antibodies

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanedi-amine), bis-diazonium derivatives (such as bis-(p-diazoniumbenzoyl)-ethyle-nediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies or to other molecules as provided herein. (See, for example, “Conjugate Vaccines”, Contributions to Microbiology and Immunology, J. M. Cruse and R. E. Lewis, Jr. (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies provided herein, to other molecules. For example, representative coupling agents can include organic compounds such as thioesters, carbodiimides, succinimide esters, diisocyanates, glutaraldehyde, diazobenzenes and hexamethylene diamines This listing is not intended to be exhaustive of the various classes of coupling agents known in the art but, rather, is exemplary of the more common coupling agents. (See Killen and Lindstrom, Jour. Immun 133:1335-2549 (1984); Jansen et al, Immunological Reviews 62:185-216 (1982); and Vitetta et al, Science 238:1098 (1987)). Preferred linkers are described in the literature. (See, for example, Ramakrishnan, S. et al, Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl)carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii)'SPDP (succinimidyl-6[3-(2-pyridyl-dithio)propionamido]hexanoate (Pierce Chem. Co., Cat #21651G); (iv) Sulfo-LC-SPDP (sulfosuccinimidyl 6[3-(2-pyridyldithio)-propianamide]hexan-oate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone. Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. F_{ab′} fragments of the antibodies provided herein can be conjugated to the liposomes as described in Martin et al, J. Biol. Chem., 257: 286-288 (1982) via a disulfide-interchange reaction.

Methods for the screening of antibodies that possess the desired specificity include, but are not limited to, enzyme linked immunosorbent assay (ELISA) and other immunologically mediated techniques known within the art.

Antibodies directed against a SARS-CoV protein (or a fragment thereof) may be used in methods known within the art relating to the localization and/or quantitation of a SARS-CoV protein (e.g., for use in measuring levels of the SARS-CoV protein within appropriate physiological samples, for use in diagnostic methods, for use in imaging the protein, and the like). In a given embodiment, antibodies specific to a SARS-CoV protein, or derivative, fragment, analog or homolog thereof, that contain the antibody derived antigen binding domain, are utilized as pharmacologically active compounds (referred to hereinafter as “Therapeutics”).

An antibody according to the invention can be used as an agent for detecting the presence of SARS-CoV (or a protein or a protein fragment thereof) in a sample. To this end, the antibody may contain a detectable label. Antibodies can be polyclonal, or preferably, monoclonal. An intact antibody, or a fragment thereof (e.g., F_{ab}, scFᵥ, or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method provided herein can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

An antibody specific for a SARS-CoV protein provided herein can be used to isolate a SARS-CoV polypeptide by standard techniques, such as immunoaffinity, chromatography or immunoprecipitation. Antibodies directed against a SARS-CoV protein (or a fragment thereof) can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies provided herein, including polyclonal, monoclonal, humanized and fully human antibodies, may be used as therapeutic agents. Such agents will generally be employed to treat or prevent a coronavirus-related disease or pathology (e.g., SARS) in a subject. An antibody preparation, preferably one having high specificity, high affinity, and/or high neutralizing potency for its target antigen, is administered to the subject and will generally have an effect due to its binding with the target. Administration of the antibody may abrogate or inhibit or interfere with the binding of the target (e.g., ACE2) with an endogenous ligand (e.g., S1 region of SARS-CoV spike protein) to which it naturally binds. In this case, the antibody binds to the target and masks a binding site of the naturally occurring ligand, thereby neutralizing SARS-CoV by inhibiting binding of S1 to ACE2.

A therapeutically effective amount of an antibody as provided herein relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment provided herein may be, by way of nonlimiting example, from about 0.1 mg/kg body weight to about 50 mg/kg body weight. Common dosing frequencies may range, for example, from twice daily to once a week.

### Pharmaceutical Compositions

In yet another embodiment, the invention comprises a pharmaceutical composition, for example, an immunogenic composition. The composition may comprise amino acids from SARS CoV spike protein and optionally, a pharmaceutically acceptable carrier.

The antibodies or agents provided herein (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Principles and considerations involved in preparing such compositions, as well as guidance in the choice of carriers or components are provided, for example, in Remington: The Science And Practice Of Pharmacy 19th ed. (Alfonso R. Gennaro, et al, editors) Mack Pub. Co., Easton, Pa., 1995, a standard reference text in the field, which is incorporated herein by reference. See also Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

Antibodies specifically binding a SARS-CoV protein or a fragment thereof provided herein, as well as other molecules identified by the screening assays disclosed herein, can be administered for the treatment of SARS-CoV-related disorders in the form of pharmaceutical compositions. Where antibody fragments are used, the smallest inhibitory fragment that specifically binds to the binding domain of the target protein is preferred. A binding fragment that also has neutralizing activity is more preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90: 7889-7893 (1993)).

The formulation can also contain more than one active compound as necessary or desirable for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The active ingredients can also be entrapped in microcapsules prepared, for example, by coacervation techniques or by interfacial polymerization, for example, hydroxymethylcellulose or gelatin-microcapsules and poly-(methylmethacrylate)microcapsules, respectively, in colloidal drug delivery systems (for example, liposomes, albumin microspheres, microemulsions, nano-particles, and nanocapsules) or in macroemulsions.

The antibodies disclosed herein can also be formulated as immunoliposomes. Liposomes containing the antibody are prepared by methods known in the art, such as described in Epstein et al, Proc. Natl. Acad. Sci. USA, 82: 3688 (1985); Hwang et al, Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

A pharmaceutical composition as provided herein is formulated to be compatible with its intended route of administration. Examples of routes of administration include parenteral, e.g., intravenous, intradermal, subcutaneous (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Optional sterilization of formulations for in vivo administration is readily accomplished by filtration through sterile filtration membranes.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous the compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms provided herein are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Screening Methods

The invention provides methods (also referred to herein as “screening assays”) for identifying modulators, i.e., candidate or test compounds or agents (e.g., peptides, peptidomimetics, small molecules or other drugs) that modulate or otherwise interfere with the binding of SARS-CoV to the SARS-CoV receptor, ACE2. Also provided are methods of indentifying compounds useful to treat SARS-CoV infection. The invention also encompasses compounds identified using the screening assays described herein.

For example, the invention provides assays for screening candidate or test compounds which modulate the interaction between SARS-CoV and its receptor, ACE2. The test compounds provided herein can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. (See, e.g., Lam, 1997. Anticancer Drug Design 12: 145).

A “small molecule” as used herein, is meant to refer to a composition that has a molecular weight of less than about 5 kD and typically less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays provided herein.

Examples of methods for the synthesis of molecular libraries can be found in the art, for example in: DeWitt, et al, 1993. Proc. Natl. Acad. Sci. U.S.A. 90: 6909; Erb, et al, 1994. Proc. Natl. Acad. Sci. U.S.A. 91: 11422; Zuckermann, et al, 1994. J. Med. Chem. 37: 2678; Cho, et al, 1993. Science 261: 1303; Carrell, et al, 1994. Angew. Chem. Int. Ed. Engl. 33: 2059; Carell, et al, 1994. Angew. Chem. Int. Ed. Engl. 33: 2061; and Gallop, et al, 1994. J. Med. Chem. 37: 1233.

Libraries of compounds may be presented in solution (see e.g., Houghten, 1992. Biotechniques 13: 412-421), or on beads (see Lam, 1991. Nature 354: 82-84), on chips (see Fodor, 1993. Nature 364: 555-556), bacteria (see U.S. Pat. No. 5,223,409), spores (see U.S. Pat. No. 5,233,409), plasmids (see Cull, et al, 1992. Proc. Natl. Acad. Sci. USA 89: 1865-1869) or on phage (see Scott and Smith, 1990. Science 249: 386-390; Devlin, 1990. Science 249: 404-406; Cwirla, et al, 1990. Proc. Natl. Acad. Sci. U.S.A. 87: 6378-6382; Felici, 1991. J. Mol. Biol. 222: 301-310; and U.S. Pat. No. 5,233,409.).

In one embodiment, a candidate compound is introduced to an antibody-angtigen complex and determining whether the candidate compound disrupts the antibody-antigen complex, wherein a disruption of this complex indicates that the candidate compound modulates the interaction between SARS-CoV and ACE2. For example, the antibody may be one of monoclonal antibodies S227.14, S230.15, or S109.8 and the antigen may be located on the S1 region of the S protein of SARS-CoV.

In another embodiment, at least one SARS-CoV protein is provided, which is exposed to at least one neutralizing monoclonal antibody. Formation of an antibody-antigen complex is detected, and one or more candidate compounds are introduced to the complex. If the antibody-antigen complex is disrupted following introduction of the one or more candidate compounds, the candidate compounds is useful to treat a SARS-CoV-related disease or disorder, e.g. SARS. For example, the at least one SARS-CoV protein may be provided as a SARS-CoV molecule, or, in another embodiment, the at least one SARS-CoV protein may be provided in a cell infected with SARS-CoV. The cell, for example, can of mammalian origin or a yeast cell.

Determining the ability of the test compound to interfere with or disrupt the antibody-antigen complex can be accomplished, for example, by coupling the test compound with a radioisotope or enzymatic label such that binding of the test compound to the antigen or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product.

In one embodiment, the assay comprises contacting an antibody-antigen complex with a test compound, and determining the ability of the test compound to interact with the antigen or otherwise disrupt the existing antibody-antigen complex. In this embodiment, determining the ability of the test compound to interact with the antigen and/or disrupt the antibody-antigen complex comprises determining the ability of the test compound to preferentially bind to the antigen or a biologically-active portion thereof, as compared to the antibody.

In another embodiment, the assay comprises contacting an antibody-antigen complex with a test compound and determining the ability of the test compound to modulate the antibody-antigen complex. Determining the ability of the test compound to modulate the antibody-antigen complex can be accomplished, for example, by determining the ability of the antigen to bind to or interact with the antibody, in the presence of the test compound.

Those skilled in the art will recognize that, in any of the screening methods disclosed herein, the antibody may be a SARS-CoV neutralizing antibody, such as monoclonal antibodies S227.14, S230.15, or S109.8. Additionally, the antigen may be a SARS-CoV protein, or a portion thereof (e.g., the S1 region of the SARS-CoV S protein). In any of the assays described herein, the ability of a candidate compound to interfere with the binding between the monoclonal antibody and the S1 region of the SARS-CoV spike protein indicates that the candidate compound will be able to interfere with or modulate the binding of SARS-CoV to the ACE2 receptor. Moreover, because the binding of the S1 protein to ACE2 is responsible for SARS-CoV entry into cells (see Li et al, Nature 426:450-54 (2003), incorporated herein by reference), such candidate compounds will also be useful in the treatment of a SARS-CoV-related disease or disorder, e.g. SARS.

The screening methods disclosed herein may be performed as a cell-based assay or as a cell-free assay. The cell-free assays provided herein are amenable to use of both the soluble form and the membrane-bound form of SARS-CoV proteins and fragments thereof. In the case of cell-free assays comprising the membrane-bound forms of the SARS-CoV proteins, it may be desirable to utilize a solubilizing agent such that the membrane-bound form of the proteins are maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamid-e, Triton® X-100, Triton® X-114, Thesit®, Isotridecypoly (ethylene glycol ether)ₙ, N-dodecyl-N,N-dimethyl-3-amm-onio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment, it may be desirable to immobilize either the antibody or the antigen to facilitate separation of complexed from uncomplexed forms of one or both following introduction of the candidate compound, as well as to accommodate automation of the assay. Observation of the antibody-antigen complex in the presence and absence of a candidate compound, can be accomplished in any vessel suitable for containing the reactants. Examples of such vessels include microtiter plates, test tubes, and micro-centrifuge tubes. In one embodiment, a fusion protein can be provided. The fusion protein adds a domain that allows one or both of the proteins to be bound to a matrix. For example, GST-antibody fusion proteins or GST-antigen fusion proteins can be adsorbed onto glutathione sepharose beads (Sigma Chemical, St. Louis, Mo.) or glutathione derivatized microtiter plates, that are then combined with the test compound, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly. Alternatively, the complexes can be dissociated from the matrix, and the level of antibody-antigen complex formation can be determined using standard techniques.

Other techniques for immobilizing proteins on matrices can also be used in the screening assays provided herein. For example, either the antibody (e.g. S227.14, S230.15, or S109.8) or the antigen (e.g. the S1 protein of SARS-CoV) can be immobilized utilizing conjugation of biotin and streptavidin. Biotinylated antibody or antigen molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, other antibodies reactive with the antibody or antigen of interest, but which do not interfere with the formation of the antibody-antigen complex of interest, can be derivatized to the wells of the plate, and unbound antibody or antigen trapped in the wells by antibody conjugation. Methods for detecting such complexes, in addition to those described above for the GST-immobilized complexes, include immunodetection of complexes using such other antibodies reactive with the antibody or antigen.

The invention further pertains to novel agents identified by any of the aforementioned screening assays and uses thereof for treatments as described herein.

### Diagnostic Assays

Antibodies provided herein can be detected by appropriate assays, e.g., conventional types of immunoassays. For example, a sandwich assay can be performed in which a SARS-CoV protein (e.g., S1, S2, and/or M) or fragment thereof is affixed to a solid phase. Incubation is maintained for a sufficient period of time to allow the antibody in the sample to bind to the immobilized polypeptide on the solid phase. After this first incubation, the solid phase is separated from the sample. The solid phase is washed to remove unbound materials and interfering substances such as non-specific proteins which may also be present in the sample. The solid phase containing the antibody of interest (e.g. monoclonal antibody S227.14, S230.15, or S109.8) bound to the immobilized polypeptide is subsequently incubated with a second, labeled antibody or antibody bound to a coupling agent such as biotin or avidin. This second antibody may be another anti-SARS-CoV antibody or another antibody. Labels for antibodies are well-known in the art and include radionuclides, enzymes (e.g. maleate dehydrogenase, horseradish peroxidase, glucose oxidase, and catalase), fluors (fluorescein isothiocyanate, rhodamine, phycocyanin, and fluorescarmine), biotin, and the like. The labeled antibodies are incubated with the solid and the label bound to the solid phase is measured. These and other immunoassays can be easily performed by those of ordinary skill in the art.

An exemplary method for detecting the presence or absence of a coronavirus (e.g. SARS-CoV) in a biological sample involves obtaining a biological sample from a test subject and contacting the biological sample with a labeled monoclonal or scFv antibody according to the invention such that the presence of the coronavirus is detected in the biological sample.

As used herein, the term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method provided herein can be used to detect SARS-CoV in a biological sample in vitro as well as in vivo. For example, in vitro techniques for detection of SARS-CoV include enzyme linked immunosorbent assays (ELISAs), Western blots, immunoprecipitations, and immunofluorescence. Furthermore, in vivo techniques for detection of SARS-CoV include introducing into a subject a labeled anti-SARS-CoV antibody. For example, the antibody can be labeled with a radioactive marker whose presence and location in a subject can be detected by standard imaging techniques.

In one embodiment, the biological sample contains protein molecules from the test subject. One preferred biological sample is a peripheral blood leukocyte sample isolated by conventional means from a subject.

The invention also encompasses kits for detecting the presence of SARS-CoV in a biological sample. For example, the kit can comprise: a labeled compound or agent capable of detecting SARS-CoV (e.g., an anti-SARS-CoV scFv or monoclonal antibody) in a biological sample; means for determining the amount of SARS-CoV in the sample; and means for comparing the amount of SARS-CoV in the sample with a standard. The compound or agent can be packaged in a suitable container. The kit can further comprise instructions for using the kit to detect SARS-CoV in a sample.

### Passive Immunization

Passive immunization has proven to be an effective and safe strategy for the prevention and treatment of viral diseases. (See Keller et al, Clin. Microbiol. Rev. 13:602-14 (2000); Casadevall, Nat. Biotechnol. 20:114 (2002); Shibata et al, Nat. Med. 5:204-10 (1999); and Igarashi et al, Nat. Med. 5:211-16 (1999), each of which are incorporated herein by reference)). Passive immunization using neutralizing human monoclonal antibodies could provide an immediate treatment strategy for emergency prophylaxis and treatment of SARS while the alternative and more time-consuming development of vaccines and new drugs in underway. Investigations with other coronaviruses have indicated that passively administered neutralizing antibodies can protect against disease (see Kolb et al, J. Virol. 75:2803 (2001)), and that it is possible to elicit neutralizing antibodies against both linear (Godet et al, J. Virol. 68:8008 (1994); Talbot et al, J. Virol. 62:3032 (1988); and Yu et al, Virology 271:182 (2000)) and conformational (see Yu et al, Virology 271:182 (2000)) epitopes of coronavirus spike proteins, and/or membrane proteins. (See Kida et al, Arch. Virol. 75:2803 (2001) and Vennema et al, Virology 181:327 (1991)). In some cases, these neutralizing antibodies have also been shown to confer protection. (See Talbot et al, 1988; Koo et al, Proc. Natl. Acad. Sci USA 96(14):7774-79 (1999); and Yu et al, 2000)).

Moreover, it has been reported that high titers of protecting IgG antibody to SARS-CoV are present in convalescent patients. Likewise, SARS patients show clinical improvement if they are given serum from previously infected patients. (see Pearson et al, Nature 424:121-26 (2003); Li et al, N. Engl. J. Med. 349:508-9 (2003)). These observations suggest that passive immunization with human monoclonal antibodies could be developed for the treatment of SARS. (See Holmes, J. Clin. Invest. 111:1605-9 (2003)).

Based on experience with other coronaviruses, those skilled in the art will recognize that a subunit vaccine can be designed to elicit neutralizing antibodies against SARS. Thus, the development of neutralizing human monoclonal antibodies and subunit vaccine candidates that are based on the epitopes on SARS-CoV spike and membrane proteins will play an important role in such therapeutic methods.

Subunit vaccines potentially offer significant advantages over conventional immunogens. They avoid the safety hazards inherent in production, distribution, and delivery of conventional killed or attenuated whole-pathogen vaccines. Furthermore, they can be rationally designed to include only confirmed protective epitopes, thereby avoiding suppressive T epitopes (see Steward et al, J. Virol. 69:7668 (1995)) or immunodominant B epitopes that subvert the immune system by inducing futile, non-protective responses (e.g. “decoy” epitopes). (See Garrity et al, J. Immunol. 159:279 (1997)).

Importantly for SARS, a subunit vaccine may circumvent the problem of antibody-dependent disease enhancement, which has been shown to occur in some other coronaviruses (see De Groot, Vaccine 21:4095-104 (2003)) and, which may be epitope dependent (see Vennema et al, Virology 181:327 (1991) and Corapi et al, J. Virol. 69:2858 (1995)). Subunit vaccines also offer potential solutions to problems including pathogen variation and hypermutability that often plague vaccine development efforts. Only epitopes from invariant, conserved regions of a pathogen's antigenic structure need be included in the subunit vaccine, thereby ensuring long-term protection for individuals and populations. Alternatively, a cocktail of peptides representing multiple variants of an antigen could be assembled, in order to mimic a range of variants of a highly mutable epitope. (See Taboga et al, J. Virol. 71:2606 (1997)). Finally, subunit vaccines are cheaper to manufacture and more stable than many other vaccine formulations.

Moreover, those skilled in the art will recognize that good correlation exists between the antibody neutralizing activity in vitro and the protection in vivo for many different viruses, challenge routes, and animal models. (See Burton, Natl. Rev. Immunol. 2:706-13 (2002); Parren et al, Adv. Immunol. 77:195-262 (2001)). The in vitro and in vivo data presented herein suggest that the human monoclonal antibodies presented herein (e.g., S227.14, S230.15, or S109.8, can be further developed and tested in in vivo animal studies to determine its clinical utility as potent viral entry inhibitors for emergency prophylaxis and treatment of SARS.

### Antigen-Ig Chimeras in Vaccination

It has been over a decade since the first antibodies were used as scaffolds for the efficient presentation of antigenic determinants to the immune systems. (See Zanetti, Nature 355:476-77 (1992); Zaghouani et al, Proc. Natl. Acad. Sci. USA 92:631-35 (1995)). When a peptide is included as an integral part of an IgG molecule, the antigenicity and immunogenicity of the peptide epitopes are greatly enhanced as compared to the free peptide. Such enhancement is possibly due to the antigen-IgG chimeras longer half-life, better presentation and constrained conformation, which mimic their native structures.

Moreover, an added advantage of using an antigen-Ig chimera is that either the variable or the F_{c} region of the antigen-Ig chimera can be used for targeting professional antigen-presenting cells (APCs). To date, recombinant Igs have been generated in which the complementarity-determining regions (CDRs) of the heavy chain variable gene (V_{H}) are replaced with various antigenic peptides recognized by B or T cells. Such antigen-Ig chimeras have been used to induce both humoral and cellular immune responses. (See Bona et al, Immunol. Today 19:126-33 (1998)).

Chimeras with specific epitopes engrafted into the CDR3 loop have been used to induce humoral responses to either HIV-1 gp120 V3-loop or the first extracellular domain (D1) of human CD4 receptor. (See Lanza et al, Proc. Natl. Acad. Sci. USA 90:11683-87 (1993); Zaghouani et al, Proc. Natl. Acad. Sci. USA 92:631-35 (1995)). The immune sera were able to prevent infection of CD4 SupT1 cells by HIV-1MN (anti-gp120 V3C) or inhibit syncytia formation (anti-CD4-D1). The CDR2 and CDR3 can be replaced with peptide epitopes simultaneously, and the length of peptide inserted can be up to 19 amino acids long.

Alternatively, one group has developed a “troybody” strategy in which peptide antigens are presented in the loops of the Ig constant (C) region and the variable region of the chimera can be used to target IgD on the surface of B-cells or MHC class II molecules on professional APCs including B-cells, dendritic cells (DC) and macrophages. (See Lunde et al, Biochem. Soc. Trans. 30:500-6 (2002)).

An antigen-Ig chimera can also be made by directly fusing the antigen with the F_{c} portion of an IgG molecule. You et al, Cancer Res. 61:3704-11 (2001) were able to obtain all arms of specific immune response, including very high levels of antibodies to hepatitis B virus core antigen using this method.

### DNA Vaccination

DNA vaccines are stable, can provide the antigen an opportunity to be naturally processed, and can induce a longer-lasting response. Although a very attractive immunization strategy, DNA vaccines often have very limited potency to induce immune responses. Poor uptake of injected DNA by professional APCs, such as dendritic cells (DCs), may be the main cause of such limitation. Combined with the antigen-Ig chimera vaccines, a promising new DNA vaccine strategy based on the enhancement of APC antigen presentation has been reported (see Casares, et al, Viral Immunol. 10:129-36 (1997); Gerloni et al, Nat. Biotech. 15:876-81 (1997); Gerloni et al, DNA Cell Biol. 16:611-25 (1997); You et al, Cancer Res. 61:3704-11 (2001)), which takes advantage of the presence of F_{c} receptors (F_{c}γRs) on the surface of DCs.

It is possible to generate a DNA vaccine encoding an antigen (Ag)-Ig chimera. Upon immunization, Ag-Ig fusion proteins will be expressed and secreted by the cells taking up the DNA molecules. The secreted Ag-Ig fusion proteins, while inducing B-cell responses, can be captured and internalized by interaction of the F_{c} fragment with F_{c}γRs on DC surface, which will promote efficient antigen presentation and greatly enhance antigen-specific immune responses. Applying the same principle, DNA encoding antigen-Ig chimeras carrying a functional anti-MHC II specific scFv region gene can also target the immunogens to all three types of APCs. The immune responses could be further boosted with use of the same protein antigens generated in vitro (i.e., “prime and boost”), if necessary. Using this strategy, specific cellular and humoral immune responses against infection of influenza virus were accomplished through intramuscular (i.m.) injection of a DNA vaccine. (See Casares et al, Viral. Immunol. 10:129-36 (1997)).

### Vaccine Compositions

Therapeutic or prophylactic compositions are provided herein, which generally comprise mixtures of one or more monoclonal antibodies or ScFvs and combinations thereof. The prophylactic vaccines can be used to prevent SARS-CoV infection and the therapeutic vaccines can be used to treat individuals following SARS-CoV infection. Prophylactic uses include the provision of increased antibody titer to SARS-CoV in a vaccination subject. In this manner, subjects at high risk of contracting SARS can be provided with passive immunity to SARS-CoV.

These vaccine compositions can be administered in conjunction with ancillary immunoregulatory agents. For example, cytokines, lymphokines, and chemokines, including, but not limited to, IL-2, modified IL-2 (Cys125.fwdarw.Ser125), GM-CSF, IL-12, γ-interferon, IP-10, MIP1β, and RANTES.

### Evaluation of Antigenic Protein Fragments (APFs) for Vaccine Potential

A vaccine candidate targeting humoral immunity must fulfill at least three criteria to be successful: it must provoke a strong antibody response (“immunogenicity”); a significant fraction of the antibodies it provokes must cross-react with the pathogen (“immunogenic fitness”); and the antibodies it provokes must be protective. While immunogenicity can often be enhanced using adjuvants or carriers, immunogenic fitness and the ability to induce protection (as evidenced by neutralization) are intrinsic properties of an antigen which will ultimately determine the success of that antigen as a vaccine component.

### Evaluation of Immunogenic Fitness

“Immunogenic fitness” is defined as the fraction of antibodies induced by an antigen that cross-react with the pathogen. (See Matthews et al, J. Immunol. 169:837 (2002)). It is distinct from immunogenicity, which is gauged by the titer of all of the antibodies induced by an antigen, including those antibodies that do not cross-react with the pathogen. Inadequate immunogenic fitness has probably contributed to the disappointing track record of peptide vaccines to date. Peptides that bind with high affinity to antibodies and provoke high antibody titers frequently lack adequate immunogenic fitness, and, therefore, they fail as potential vaccine components. Therefore, it is important to include immunogenic fitness as one of the criteria for selecting SARS vaccine candidates.

A common explanation for poor immunogenic fitness is the conformational flexibility of most short peptides. Specifically, a flexible peptide may bind well to antibodies from patients, and elicit substantial antibody titers in naive subjects. However, if the peptide has a large repertoire of conformations, a preponderance of the antibodies it induces in naive subjects may fail to cross-react with the corresponding native epitope on intact pathogen.

Like short peptides, some APFs may be highly flexible and, therefore may fail as vaccine components. The most immunogenically fit APFs are likely to consist of self-folding protein subdomains that are intrinsically constrained outside the context of the whole protein.

Because immunogenic fitness is primarily a property of the APF itself, and not of the responding immune system, immunogenic fitness can be evaluated in an animal model (e.g. in mice) even though ultimately the APF will have to perform in humans.

The immunogenic fitness achieved by APFs is evaluated by immunosorption of anti-APF sera with purified spike or membrane protein, in a procedure analogous to that described in Matthews et al, J. Immunol. 169:837 (2002). IgG is purified from sera collected from mice that have been immunized. Purified, biotinylated spike and membrane proteins (as appropriate, depending on the particular APF with which the mice were immunized) are mixed with the mouse IgG and incubated. Streptavidin-coated sepharose beads are then added in sufficient quantity to capture all of the biotinylated spike or membrane protein, along with any bound IgG. The streptavidin-coated beads are removed by centrifugation at 13,000 rpm in a microcentrifuge, leaving IgG that has been depleted of antibodies directed against the spike or membrane protein, respectively. Mock immunosorptions are performed in parallel in the same way, except that biotinylated BSA will be substituted for SARS protein as a mock absorbent.

To measure the immunogenic fitness of APFs, the spike- or membrane-absorbed antibodies and the mock-absorbed antibodies are titered side-by-side in ELISA against the immunizing APF. For APFs affinity selected from a phage display NPL, the antigen for these ELISAs will be purified APF-GST fusion proteins. For the potentially glycosylated APFs from the mammalian cell display NPL, the antigen for these ELISAs will be APF-F_{c} fusion proteins secreted by mammalian cells and purified with protein A. The percentage decrease in the anti-APF titer of spike- or membrane-absorbed antibodies compared with the mock-absorbed antibodies will provide a measure of the immunogenic fitness of the APF.

### Methods of Treatment

The invention provides for both prophylactic and therapeutic methods of treating a subject at risk of (or susceptible to) a coronavirus-related disease or disorder. Such diseases or disorders include, but are not limited to, e.g., SARS.

### Prophylactic Methods

In one aspect, the invention provides methods for preventing a coronavirus-related disease or disorder in a subject by administering to the subject a monoclonal antibody or scFv antibody provided herein or an agent identified according to the methods provided herein.

Subjects at risk for coronavirus-related diseases or disorders include patients who have come into contact with an infected person or who have been exposed to the coronavirus in some other way. Administration of a prophylactic agent can occur prior to the manifestation of symptoms characteristic of the coronavirus-related disease or disorder, such that a disease or disorder is prevented or, alternatively, delayed in its progression.

The appropriate agent can be determined based on screening assays described herein. Alternatively, or in addition, the agent to be administered is a scFv or monoclonal antibody that neutralizes SARS that has been identified according to the methods provided herein.

### Therapeutic Methods

Another aspect of the invention pertains to methods of treating a coronavirus-related disease or disorder in a patient. In one embodiment, the method involves administering an agent (e.g., an agent identified by a screening assay described herein and/or a scFv antibody or monoclonal antibody identified according to the methods provided herein), or combination of agents that neutralize the coronavirus to a patient suffering from the disease or disorder.

In certain embodiments, a method of treating SARS in a patient is provided, said method comprising administering at least one monoclonal antibody, or a fragment thereof, selected from the group consisting of S227.14, S230.15, and S109.8.

In further embodiments, two or more of said monoclonal antibodies or fragments thereof, are administered together to said patient.

In certain embodiments, said antibody or fragment thereof can cross-neutralize human and zoonotic SARS-CoV strains.

In certain embodiments, said antibody or fragment thereof is administered within the first 24 hours following SARS-CoV infection.

In certain embodiments, said antibody is administered with an agent that enhances bidirectional IgG transport across epithelial barriers mediated in part by MHC class I-related F_{c}.

The invention will be further described in the following examples, which do not limit the scope of the invention described in the claims.

### EXAMPLES

### Example 1

### General Methods Employed in Assays

Viruses and cells. The generation and characterization of the recombinant infectious clone (ic) of Urbani, icCUHK-W1, icGZ02, icHC/SZ/61/03, icA031G and icMA15 have been described previously (35, 39). Briefly, the Urbani spike gene in icUrbani was replaced by the various spike genes of CUHK-W 1, GZ02, HC/SZ/61/03 and A031G. All recombinant icSARS-CoV strains were propagated on Vero E6 cells in Eagle's minimal essential medium (Invitrogen, Carlsbad, Calif.) supplemented with 10% fetal calf serum (HyClone, Logan, Utah), kanamycin (0.25 μg/ml) and gentamycin (0.05 μg/ml) at 37° C. in a humidified CO₂ incubator. All work was performed in a biological safety cabinet in a biosafety level 3 (BSL3) laboratory containing redundant exhaust fans. Personnel were equipped with powered air-purifying respirators with high-efficiency particulate air and organic vapor filters (3M, St. Paul, Minn.), wore Tyvek suits (DuPont, Research Triangle Park, N.C.) and were double gloved.

Human monoclonal antibodies. Human mAbs against SARS-CoV were generated as described previously in WO 04076677A2. EBV-transformed B cells are screened for those producing antibodies of the desired antigen specificity, and individual B cell clones can then be produced from the positive cells.

The screening step may be carried out by ELISA, by staining of tissues or cells (including transfected cells), a neutralization assay or one of a number of other methods known in the art for identifying desired antigen specificity. The assay may select on the basis of simple antigen binding, or may select on the additional basis of a desired function e.g. to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

The cloning step for separating individual clones from the mixture of positive cells may be carried out using limiting dilution, micromanipulation, single cell deposition by cell sorting or another method known in the art. In certain embodiments, the cloning may be carried out using limiting dilution.

The mAbs were initially screened for their binding capacity to SARS-CoV S expressing cells and subsequently tested for their ability to neutralize the Frankfurt isolate of the SARS-CoV (AY310120). A panel of 23 SARS-CoV S specific mAbs and a control mAb (D2.2) specific for diphtheria toxin, were used for further study.

Neutralization assay. Mab neutralizing titers were determined by either micro neutralization assay or plaque reduction neutralization titer assay (PRNT50%) (39). For the micro neutralization assay, mAbs were serially diluted two-fold, and incubated with 100 pfu of the different icSARS-CoV strains for 1 h at 37° C. Virus and antibodies were then added to a 96-well plate with 5×10³ Vero E6/well in 5 wells per antibody dilution. Wells were checked for cytopathic effect (CPE) at 4-5 days post infection and 50% neutralization titer was determined as the mAb concentration at which at least 50% of wells showed no CPE. For the PRNT50%, mAbs were serially diluted two-fold, and incubated with 100 pfu of the different icSARS-CoV strains for 1 h at 37° C. Virus and antibodies were then added to a 6-well plate with 5×10⁵ Vero E6/well in duplicate. After a 1 h incubation period at 37° C., cells were overlayed with 3 ml of 0.8% agarose in media. Plates were incubated for 2 days at 37° C., stained with neutral red for 3 h and plaques were counted. The percentage of neutralization was calculated as: 1-(number of plaques with antibody/number of plaques without antibody)×100%. All assays were performed in duplicate. Importantly, a good correlation has been noted between the two assays (data not shown).

Inhibition of binding of SARS-CoV spike glycoprotein to ACE-2. Serial dilutions of mAbs in PBS-1% FCS were incubated for 20 min at 4° C. with 5 μg/ml SARS-CoV S glycoprotein (S1 domain amino acids 19-713 of WH20 isolate [99.8% amino acid homology with Urbani; AY772062) fused to the F_{c} region of human Ig (Aalto Bio Reagents, Dublin, Ireland). The mixture was added to a single cell suspension of 4×10⁴ ACE-2-transfected DBT cells that had been sorted for stable and relatively uniform levels of ACE2 expression. After 20 min the cells were washed and stained with PE-conjugated F(ab′)₂ fragments of a goat anti human F_{c γ} specific antibody (Jackson Immunoresearch Laboratories). The percentage of binding inhibition was calculated accordingly to the following formula where the Bₘₐₓ is represented by the average of six wells: (1−(% positive events of the sample/Bₘₐₓ %)×100%. The concentration of the antibody needed to achieve 50% of binding inhibition (IC50) was calculated with GraphPad Prism software using a non-linear regression fitting with variable slope.

Detection of human mAbs. Reactivity of mAbs with native or denatured Urbani S recombinant protein was determined by ELISA. Briefly, 96 well plates were coated with 1 μg/ml of recombinant Urbani S glycoprotein (NR-686; NIH Biodefense and Emerging Infections Research Resources Repository, NIAID, NIH). Wells were washed and blocked with 5% non-fat milk for 1 h 37° C. and incubated with serially diluted mAbs for 1.5 h at 37° C. Bound mAbs were detected by incubating alkaline-phosphatase-conjugated goat anti-human IgG (A-1543; Sigma) for 1 h at 37° C. and developed by 1 mg/ml p-nitrophenylphosphate substrate in 0.1 M glycine buffer (pH 10.4) for 30 min at room temperature. The optical density (OD) values were measured at a wavelength of 405 nm in an ELISA reader (Bio-Rad Model 680).

Competition for binding to SARS-CoV S glycoprotein. MAbs were purified on Protein G columns (GE Healthcare) and biotinylated using the EZ-Link NHS-PEO solid phase biotinylation kit (Pierce). An ELISA assay was used as described above to measure the competition between unlabeled and biotinylated mAbs for binding to immobilized SARS-CoV S glycoprotein. Unlabelled competitor mAbs were added at 5 μg/ml. After 1 h biotinylated mAbs were added at a limiting concentration (0.1 μg/ml) that was chosen to give a net optical density in the linear part of the titration curve, allowing inhibitory effects of the unlabelled mAb to be quantitated. After incubation for 1 h, the plates were washed and the amount of biotinylated mAb bound was detected using alkaline phosphatase-labeled streptavidin (Jackson Immunoresearch). The percentage of inhibition was calculated with the means of triplicate tests using the following formula

(1−[(ODₛₐₘₚₗₑ−OD_{neg ctr})/(OD_{pos ctr}−OD_{neg ctr})])×100%.

Escape mutant analysis. Neutralization resistant SARS-CoV mutants were generated as described previously (Rockx et al, 2007, J. Virol. 81: 7410-7423). Briefly, 1×10⁶ pfu of icUrbani and GZ02 were incubated with 30 μg of a neutralizing mAb and then inoculated onto cells in the presence of mAb. The icHC/SZ/61/03 isolate was used for generating a neutralization escape mutant for mAb S227.14, as several attempts to generate escape mutants from this antibody using icUrbani or icGZ02 proved unsuccessful. The development of cytopathic effect (CPE) was monitored over 72 hrs and progeny viruses harvested. MAb treatment was repeated two additional times with more rapid CPE noted with each passage. Passage 3 viruses were plaque purified in the presence of mAb and neutralization resistant viruses were isolated. The S gene of at least two individual plaques was sequenced as previously described (34) and the neutralization titers between wild type and mAb-resistant viruses were determined as described above.

Structural Analyses. The crystal structure coordinates of SARS-CoV RBD interacting with the human ACE-2 receptor (PDB code 2AJF) (23) were used as a template to generate each set of mutations using the Rosetta Design web server (http://rosettadesign.med.unc.edu/). In each case, the SARS-CoV RBD structure was analyzed using the molecular modeling tool, MacPyMol (DeLano Scientific), to determine which amino acid residues were proximal to the amino acid being targeted for replacement. Briefly, each amino acid to be altered was highlighted and all other amino acid residues within an interaction distance of 5 Å were identified. Using the Rosetta Design website, the amino acid replacements were incorporated and all amino acid residues within the 5 Å interaction distance were relaxed to allow the program to repack the side chains to an optimal energetic state. This process was repeated with each mutation and series of mutations. Ten models were generated for each set of mutations, and the best model was selected based on the lowest energy score and further evaluated using Mac Pymol. In all cases, the lowest energy score was identical between several of the predicted models, suggesting an optimal folding energy of the chosen model.

Passive immunization. Female BALB/cAnNHsd mice (10-week-old or 12-month-old from Harlan, Indianapolis, Ind.) were anesthetized with a ketamine (1.3 mg/mouse) xylazine (0.38 mg/mouse) mixture administered intraperitoneally in a 50 μl volume. Each mouse was intranasally inoculated with 10⁶ pfu (icUrbani, icGZ02 or icHC/SZ/61/03) or 10⁵ pfu (icMA15) of icSARS in a 50 μl volume. Table 2 below summarizes the passive immunization studies performed.

TABLE 2
Experimental design of passive immunization studies in mice.

| | μg | | Day of | Challenge | | |
|---|---|---|---|---|---|---|
| Experiment | mAb | mAb | vaccination | virus | Age of mice | |
| 1 | 25 | D2.2, S109.8, S227.14, S230.15 | −1 | icUrbani, icGZ02, icHC/SZ/61/03 | 12 | months |
| 2 | 250 | D2.2, S109.8, S227.14, S230.15 | −1 | icUrbani, icGZ02, icHC/SZ/61/03 | 12 | months |
| 3 | 250 | S109.8 + S227.14 + S230.15 (Cocktail) | −1 | icHC/SZ/61/03 | 12 | months |
| 4 | 250 | S230.15 | −1 | icHC/SZ/61/03 | 10 | weeks |
| 5 | 25 | D2.2, S109.8, S227.14, S230.15 | −1 | icMA15 | 10 | weeks |
| 6 | 250 | S230.15 | −1, 0, 1, 2, 3 | GZ02 | 12 | months |

In experiment 1 and 2 (Table 2), 12-month-old mice were injected intraperitoneally with 25 or 250 μg of various human mAbs (D2.2, S109.8, S227.14 or S230.15) in a 400 μl volume at 1 day prior to intranasal inoculation with 10⁶ pfu of the different icSARS-CoV strains (n=3 per mAb, per virus, per time point). In experiment 3 (Table 2), 12-month-old mice were injected with a cocktail of S109.8, S227.14 and S230.15 (cocktail with 83 μg of each mAb) with a total concentration of 250 μg mAb in 400 μl at 1 day prior to inoculation with 10⁶ pfu icHC/SZ/61/03 (n=3 per time point). In experiment 4 (Table 2), 10-week-old mice were injected with 250 μg of S230.15 at 1 day prior to inoculation with 10⁶ pfu of icHC/SZ/61/03 (n=4). In experiment 5 (Table 2), 10-week-old mice were injected with 25 μg of D2.2, S109.8, S227.14 or S230.15 at 1 day prior to inoculation with 10⁵ pfu of icMA15 (n=3 per mAb, per time point). In experiment 6 (Table 2), 12-month-old mice were injected with 250 μg of S230.15 at −1, 0, 1, 2 or 3 days post inoculation with 10⁶ pfu icGZ02 (n=5 per treatment, per time point). All animals were weighed daily and 2, 4 or 5 days post infection serum and lung samples were removed and frozen at −70° C. for later plaque assay determination of viral titers. Lung tissue was also removed for histological examination on day 4 or five depending on whether animals had to be euthanized due to >20% weight loss.

Virus titers in lung samples. Tissue samples were weighed and homogenized in 5 equivalent volumes of PBS to generate a 20% solution. The solution was centrifuged at 13,000 rpm under aerosol containment in a table top centrifuge for 5 min, the clarified supernatant serially diluted in PBS, and 200 μl volumes of the dilutions placed onto monolayers of Vero cells in 6-well plates. Following a 1-hour incubation at 37° C., cells were overlaid with 0.8% agarose containing medium. Two days later, plates were stained with neutral red and plaques counted.

Histology. All tissues were fixed in 4% PFA in PBS (pH 7.4) prior to paraffin embedding, sectioning at 5 μm thickness, and hematoxylin and eosin staining. Lung pathology was evaluated in a blinded manner.

### Example 2

Identification of cross-neutralizing mAb. A panel of 23 human mAbs was tested for their neutralizing activity against one or multiple icSARS-CoV bearing spike variants from the late, middle, early and zoonotic phases of the epidemic. The panel includes a number of mAbs (S228.11, S222.1, S237.1, S223.4, S225.12, S226.10, S231.19, S232.17, S234.6, S227.14, S230.15, S110.4, S111.7) that were not described in isolation (49) and with the exception of S110.4 and S111.7, were all isolated at a late time point after infection with SARS-CoV (2 years).

All mAbs efficiently neutralized the late phase icUrbani isolate (Table3) which was homologous to the strain isolated from the patient used to produce the mAbs (52). Interestingly, when testing the mAbs against the middle, early and zoonotic isolates, six distinct neutralization patterns were identified (Table 3). Two unique group I monoclonal antibodies were identified that specifically neutralized the homologous late phase isolate, icUrbani. Two monoclonal antibodies comprised group II, which neutralized the homologous icUrbani strain about 10 fold more efficiently than the middle phase isolate, icCUHK-W1. Group III contained five monoclonal antibodies that were about 50 fold more efficient at neutralizing the reference icUrbani strain as compared with the group I antibodies. These antibodies were extremely efficient at neutralizing the human late, middle and early phase isolates (n=5) but not the zoonotic isolates at all concentrations tested (8 ng/ml to 16 μg/ml). Group IV consists of 8 mAbs that were extremely efficient in neutralizing the human isolates as well as the palm civet isolate icHC/SZ/61/03. It is likely that two or more neutralizing epitopes exist within this cluster as some mAbs were equally efficient at neutralizing human and zoonotic isolates (e.g. 225.12, 226.10, 234.6) while others required 10 fold antibody to neutralize the civet isolate (e.g. 218.9, 231.19, 232.17). The group V cluster consisted of two mAbs that neutralized variable subsets of the human and zoonotic strains but only at high concentrations. Finally group VI consisted of four mAbs that neutralized all human and zoonotic strains available within our panel of variant SARS-CoV spike variants. Because of the varying concentrations of antibody needed to neutralize isolates for each monoclonal antibody in group VI, we suspected that at least two or three different pan specific neutralizing epitopes likely exist in the SARS-CoV S glycoprotein. See results in Table 3, below.

### Example 3

Identification of mAbs that inhibit binding of SARS-CoV S glycoprotein to ACE-2. To identify the mAbs that directly inhibit the binding of SARS-CoV to its cellular receptor ACE-2 as a mechanism of neutralization, we assessed the capacity of the mAb panel described above to inhibit the binding of the SARS-CoV S1 domain to human ACE-2 expressed on the surface of a transfected murine DBT cell line. The antibody activity is expressed as the concentration that blocks 50% of spike binding to ACE-2 as well as the maximum inhibition values (Table 3). Most of the antibodies completely inhibited binding, although with different potencies (Table 3; see for example S230 and S3.1). Of note, some antibodies only partially inhibited binding of the spike protein even when tested at the highest concentrations (see for example S124.6, S109.8). Not surprisingly a significant correlation was observed between neutralization titers and inhibition titers of SARS-CoV S glycoprotein binding to ACE-2 (r²=0.344; p=0.002). However a few antibodies such as S3.1 and S127.6 showed a high viral neutralization capacity in spite of a low capacity to interfere with spike binding to its receptor (Table 3).

Results of the SARS human mAbs cross-neutralization and SARS-CoV/ACE-2 binding inhibition assays are shown in the following Table 3.

TABLE 3

| | | | | | | | | | | Inhibition of SARS-CoV S | |
|---|---|---|---|---|---|---|---|---|---|---|---|
| | | 50% Neutralization titer (ng/ml) | | | | | | | | binding to ACE-2 | |
| Group | mAb | Urbani | CUHK-W1 | GZ02 | HC/SZ/61/03 | A031G | GZ02-109-1 | GZ02-109-2 | GZ02-230 | % inhibition | IC50 (ng/ml) |
| I | 132 | 1984 | — | — | — | — | nt | nt | nt | 60 | 2570 |
| | 228.11 | 196 | — | — | — | — | nt | nt | nt | 97 | 598 |
| II | 111.7 | 154 | 1232 | — | — | — | nt | nt | nt | 96 | 1208 |
| | 224.17 | 194 | 1552 | — | — | — | nt | nt | nt | 98 | 297 |
| III | 3.1 | 45 | 180 | 720 | — | — | nt | nt | nt | 96 | 868 |
| | 127.6 | 65 | 259 | 518 | — | — | nt | nt | nt | 97 | 876 |
| | 217.4 | 30 | 59 | 118 | — | — | nt | nt | nt | 99 | 114 |
| | 222.1 | 51 | 202 | 808 | — | — | nt | nt | nt | 98 | 98 |
| | 237.1 | 8 | 67 | 34 | — | — | nt | nt | nt | 97 | 66 |
| IV | 110.4 | 81 | 322 | 644 | 1288 | — | nt | nt | nt | 99 | 476 |
| | 218.9 | 31 | 123 | 246 | 1968 | — | nt | nt | nt | 101 | 280 |
| | 223.4 | 20 | 79 | 158 | 316 | — | nt | nt | nt | 99 | 112 |
| | 225.12 | 9 | 18 | 72 | 72 | — | nt | nt | nt | 99 | 68 |
| | 226.10 | 23 | 90 | 360 | 180 | — | nt | nt | nt | 99 | 92 |
| | 231.19 | 18 | 71 | 141 | 2256 | — | nt | nt | nt | 99 | 120 |
| | 232.17 | 90 | 180 | 360 | 2880 | — | nt | nt | nt | 100 | 95 |
| | 234.6 | 64 | 2032 | 254 | 254 | — | nt | nt | nt | 100 | 142 |
| V | 124.5 | 1400 | 5600 | — | 1120 | 5600 | nt | nt | nt | 56 | 4700 |
| | 219.2 | 248 | 992 | — | — | 496 | nt | nt | nt | 44 | >3000 |
| VI | 109.8 | 424 | 848 | 3392 | 424 | 53 | — | — | 3300 | 85 | 525 |
| | 215.17 | 25 | 100 | 200 | 400 | 3200 | nt | nt | nt | 98 | 200 |
| | 227.14 | 19 | 77 | 153 | 306 | 77 | 150 | 150 | 150 | 100 | 126 |
| | 230.15 | 20 | 40 | 160 | 160 | 80 | 155 | 155 | — | 99 | 84 |

Table 3 Legend. Characterization of a panel of human mAbs for their capacity to neutralize human and zoonotic SARS-CoV strains and inhibit SARS-CoV S glycoprotein binding to human ACE-2. A panel of 23 human mAbs were tested for their capacity to neutralize recombinant SARS-CoV S glycoprotein variants (Urbani, CUHK-W1, GZ02, HC/SZ/61/03 and A031G) and neutralization escape variants (GZ02-109-1, GZ02-109-2 and GZ02-230) by human mAbs were determined MAbs are ranked in 6 groups according to their capacity to neutralize different SARS-CoV S glycoprotein variants. The mAb concentration, at which 50% of the viruses is neutralized, is shown (ng/ml). In addition the percentage (%) of maximal inhibition of SARS-CoV S glycoprotein binding to human ACE-2, expressed by murine DBT cells, by the mAbs is shown along with the concentration at which 50% of the binding is blocked (IC50). “−” no neutralizing titer detected; “nt:” not tested.

### Example 4

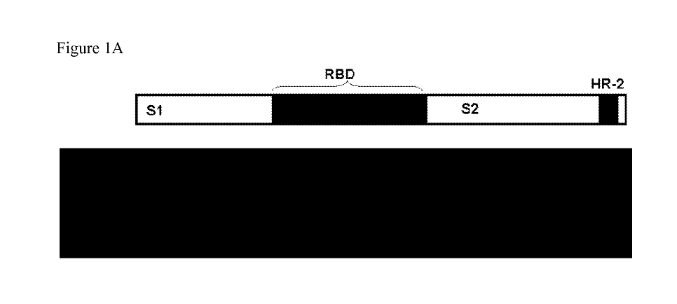

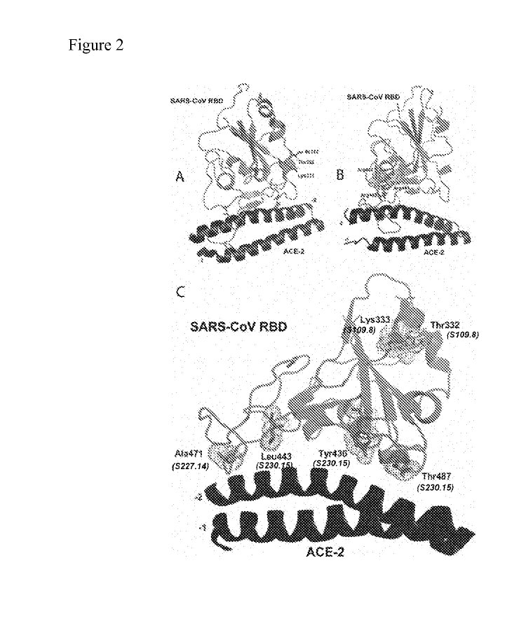

Phylogenetic analysis of viral neutralization. By using a panel of S glycoprotein variants, the amino acid changes associated with loss of neutralization can be identified. To identify possible locations of neutralizing epitopes recognized by these mAbs, the neutralization groups were annotated in accordance with the amino acid sequences variation noted in the different S glycoproteins used in this study (FIG. 1A). Interestingly group I mAbs S132 and S228.11 uniquely neutralized icUrbani which differs at positions G77D and I244T in the S1 domain from the resistant middle phase isolate icCUHK-W1 (FIG. 1A). Although the mechanism is unclear, these two unique residues in icUrbani either individually or in concert result in a) micro variation within overlapping epitopes, b) changes in conformational epitopes, or c) mutations which alter the surface topology of a group I epitope. Consonant with these findings, four amino acid changes (FIG. 1A) were observed between the middle phase icCUHK-W1 and the early phase icGZ02 S glycoprotein. The fact that group II antibodies efficiently inhibit RBD binding to ACE-2 implies that the critical residues are likely those residing within the RBD (e.g. G311R and K344R). In contrast, the mutations that influence the binding and activity of the group III mAbs are the most complex and influenced by one or more of 15 amino acid changes between the early icGZ02 and the zoonotic palm civet icHC/SZ/61/03 isolate. These changes are scattered throughout the S1, RBD and S2 domains (FIG. 1A), however all group III antibodies efficiently inhibit RBD binding to ACE-2 suggesting that the critical residues are those residing within the RBD. The RBD residues include F360, L472, N479 and D480. The neutralization activity of the group IV mAbs cluster is heavily influenced by two amino acid changes between the zoonotic strains icHC/SZ/61/03 and the raccoon dog isolate, icA031G, located in the RBD (P462S) or in an S2 (E821Q) domain of the S glycoprotein (FIG. 1A). Again, the efficient inhibition of RBD binding to the ACE-2 suggests that the P462S is the critical residue. The recognition domain of the group VI broad spectrum antibodies must be conserved across the panel and the location is unclear, although S230.15 has been previously shown to bind to the RBD in the S glycoprotein (60) by competition ELISA and all the group VI mAbs have been shown to interfere with the binding to ACE-2 expressed on the surface of the cell membrane.

### Example 5

Competition studies for the definition of epitopes recognized by broadly neutralizing mAbs. Our data suggests that the majority of the human mAbs recognize epitopes differentially defined by a few mutations within the RBD. To provide a more thorough understanding of the binding domains recognized by different monoclonal antibodies, competition studies were performed to determine the spatial proximity of each of the neutralizing epitopes recognized by representative mAbs. Several mAbs, S109.8, S227.14 and S230.15 (group VI), were biotinylated and tested for their capacity to bind the SARS-CoV S glycoprotein in the presence of other unlabeled mAbs. In interpreting competition results, it should be taken into account that when two epitopes overlap, or when the areas covered by the arms of the two mAbs overlap, competition should be almost complete. Weak inhibitory or enhancing effects may simply reflect a decrease in affinity owing to steric or allosteric effects (29, 51). The two most potent cross-neutralizing mAbs S227.14 and S230.15 compete with each other (FIG. 1B) and with several other mAbs with the exception of the group I mAbs (S138 and S228.11), group V mAbs (S124.5 and S219.2), S3.1 (group III) and S109.8 (group VI). The S230.15 mAb has a higher affinity than the S227.14 mAb since it competes with the S227.14 mAb at a 16 fold lower concentration than that required for the S227.14 mAb to compete with S230.15 (46 ng/ml and 738 ng/ml, respectively). The S109.8 mAb did not compete with any of the mAbs, although limited inhibition was seen with S127.6 (61%; FIG. 1B).

### Example 6

Escape mutant analysis of neutralizing mAbs. We previously used the icGZ02 isolate to successfully generate neutralization escape mutants for two broadly neutralizing mAbs S109.8 and S230.15, which selected for escape mutations at positions T332I or K333N, and L443R respectively (Rockx et al, 2008, J. Virol. 82: 3220). However, the use of this isolate limits the number of mAbs that could be used for these escape analyses. Therefore, the icUrbani isolate was used to generate antibody neutralization escape mutants by incubating and culturing high titers of virus in the presence of selected mAbs chosen from the five distinct neutralization groups previously described by our group. After 3 passages, the resulting viruses were plaque purified and 2 plaques of each virus were sequenced to identify the amino acid changes associated with the antibody escape phenotype.

The S109.8 escape mutant of icGZ02 was no longer neutralized by S109.8 compared to the wild type (WT) icGZ02 even at antibodies exceeding 20 μg/ml (Table 3). However both S227.14 and S230.15 were equally effective at neutralizing the S109.8 escape mutant of icGZ02 as compared to the WT.

Similarly the S230.15 escape mutant was no longer neutralized by S230.15 but was still effectively neutralized by both S109.8 and S227.14 mAbs (Table 3). This was particularly interesting since S227.14 was shown to compete with S230.15 for binding to the RBD confirming that both S227.14 and S230.15 recognize overlapping but distinct epitopes. The generation of a mAb neutralization escape variant using mAb S227.14 was unsuccessful after two independent attempts using icGZ02; the usage of the icUrbani isolate was also unsuccessful. Reasoning that the RBD backbones of these two closely related viruses were insufficiently flexible to allow for the emergence of escape variants, we selected for variants using the icHC/SZ/61/03 isolate. Two mAb neutralization escape mutants were isolated which contained a single amino acid mutation at position 390, resulting in either a K390Q or K390E change. Of note, both S227.14 escape mutants were still neutralized by S230.15 and S109.8.

A minimum of 2 plaques of each escape variant were sequenced to identify mutations associated with the antibody escape phenotype. All 5 plaques of the S230.15 escape mutant contained a single amino acid change at location L443R. Four out of six plaques of the S109.8 escape mutants contained a single amino acid change at T332I while two plaques contained a single amino acid change in an adjacent residue at position K333N.

### Example 7

Structural modeling of cross-neutralizing epitopes. Recently the structure of the SARS-CoV RBD complexed with its receptor ACE2 was resolved, allowing for structural modeling of amino acid changes within the RBD. Both mutations observed with the S109.8 escape mutants flank the side of the RBD in a loop that is not in direct contact with the receptor, ACE2 (FIG. 2A). The T332I change results in a protrusion from the surface due to the additional CH3 group as well as becoming strongly hydrophobic. Alternatively, the amino acid change from Lys to Asn at position 333 removes a positive charge. Both mutations clearly affect binding of the S109.8 mAb. The mechanism of neutralization by S109.8 is unknown but may either involve structural changes to the RBD after binding or provide steric hindrance that antagonizes receptor binding in some unspecified manner.

Structural analysis of the S230.15 escape mutant showed that subtle remodeling of the receptor binding pocket did not impact binding of the ACE2. The selected arginine mutation residue is likely forced into the binding pocket by surrounding positive charged amino acids. At this site, a binding pocket exists that can accommodate the larger side chain without disrupting interface site interactions (FIG. 2B). However, the presence of arginine at this position likely ablates binding of S230.15. These data support the hypothesis that the S230.15 mAb neutralizes SARS-CoV by directly blocking the interaction with its receptor ACE2.

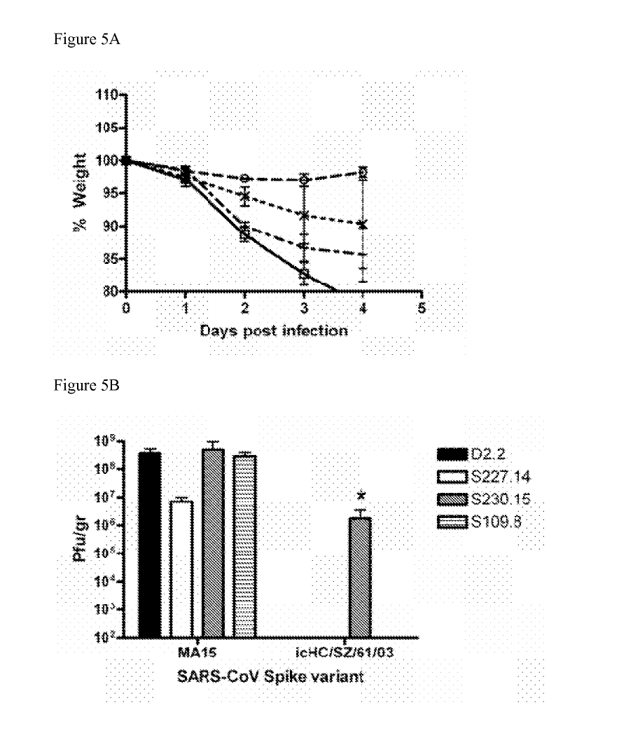

The combined results from the phylogenetic analysis, competition assays, and escape mutant analysis allowed us to identify the amino acid that were associated with the neutralization efficacy of the different cross-neutralizing mAbs. By mapping the location of these amino acids onto the crystal structure of the SARS-CoV Urbani strain RBD bound to ACE2, putative locations of the cross-neutralizing epitopes could be identified (FIG. 2C). S230.15 likely recognizes an epitope that includes amino acid 443, as shown by escape variant analysis, as well as amino acid 487, as shown by reduced in vitro neutralization of an SZ16 spike variant with a T487S change (60), and amino acid 436, as shown by reduced in vivo protection against icMA15 (FIG. 5A). The epitope recognized by the S227.14 mAb partially overlaps with that of S230.15 but is not affected by the L443R change identified in the S230.15 escape mutant. In addition, the cross-neutralization data suggests that the amino acid change K390Q/E associated with mAb neutralization escape from S227.14 is uniquely separate from other escape mutants. The mutation resides in close proximity (within 4 Å) to residue 491 which has been shown to interact with multiple residues on the ACE2 molecule. It is likely that the close proximity of the mAb S227.14 binding site to this RBD residue that engage the ACE2 receptor prevents S-ACE2 interaction. Alternatively, the antibody may allow for binding but prevent downstream steps in entry. Finally, the epitope recognized by S109.8 includes amino acid 332 and 333 as shown by escape mutant analysis.

### Example 8

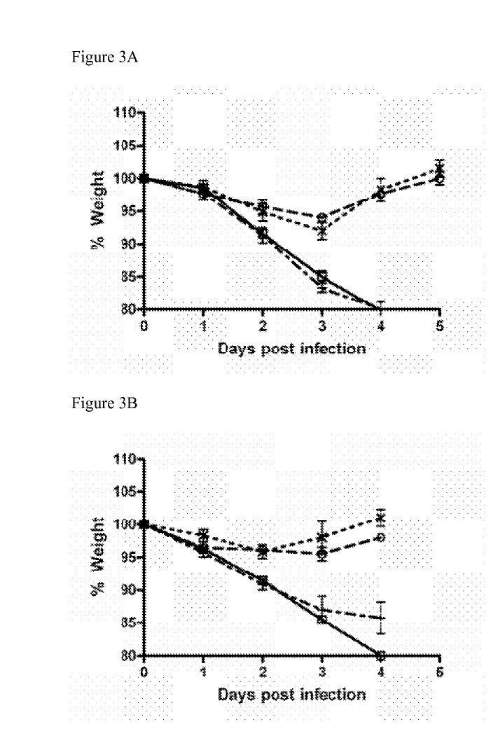

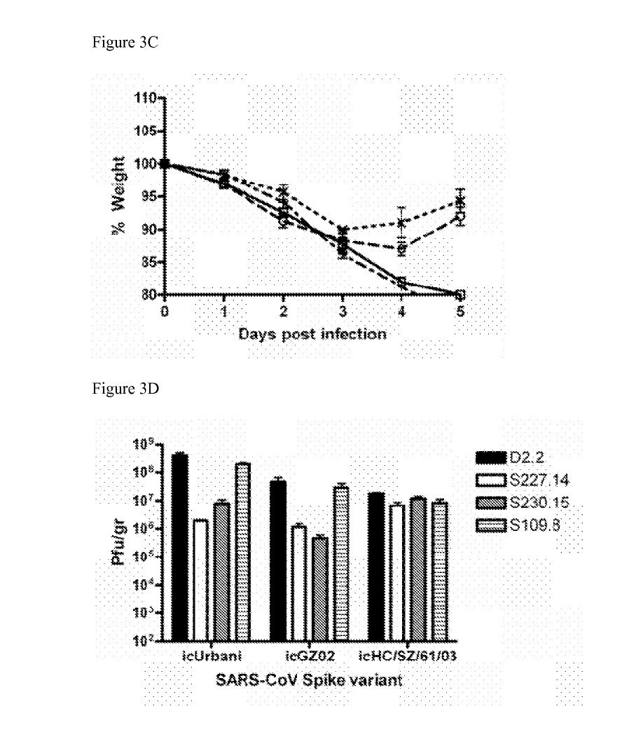

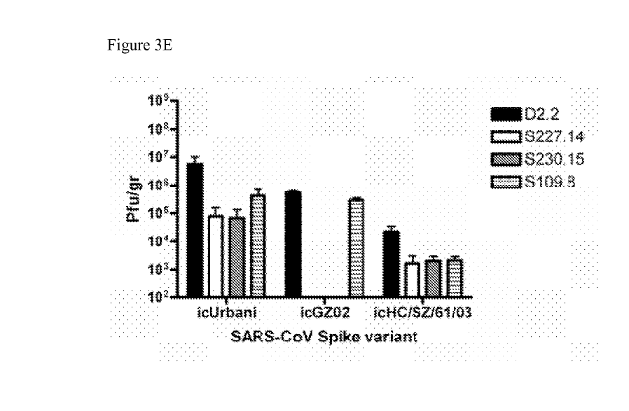

Human mAb as prophylaxis in Senescent Models. Our data strongly supports the hypothesis that S109.8, S227.14 and S230.15 are potent cross-neutralizing human mAbs that recognize the RBD of the SARS-CoV S glycoprotein. S109.8 recognizes a unique epitope distinct from the receptor binding site while S227.14 and S230.15 recognize partially overlapping epitopes that coincide with the receptor binding site. These broad spectrum neutralizing monoclonal antibodies were therefore tested for their ability to protect against lethal homologous and heterologous SARS-CoV challenge in vivo. Previous studies in an acute non-lethal murine model indicated that 200 μg of S230.15 mAb was protective against SARS-CoV infection while mAb prophylaxis has not been studies in aged mice (60). SARS-CoV typically produces severe disease in senescent populations, requiring a prophylactic approach that would protect young and older populations. We have previously shown that infection of 12-month-old BALB/cBy mice with 10⁵ pfu of icGZ02 or icHC/SZ/61/03 resulted in death or >20% weight loss by day 4 or 5 (39), whereas mice infected with 10⁵ pfu of icUrbani lost only 10% weight. Interestingly by increasing the challenge titer 10-fold to 10⁶ pfu, the typically mild pathogenic phenotype of icUrbani was increased as weight loss approached 20% by day 4 or 5 in 1 year old BALB/cAnNHsd (FIG. 3A; D2.2).

Twelve-month-old BALB/c mice that received 25 μg S227.14 or S230.15 intraperitoneally 24 hrs prior to infection, were protected against significant weight loss (t-test; p<0.01) and had reduced viral titers in their lungs on 2 days and 5 days, approaching 1.5-2 log and 2-4 log reduction respectively, following challenge with icUrbani or icGZ02 (FIGS. 3A, B, D and E). Animals challenged with icHC/SZ/61/03 that had received 25 μg of the S227.14 or S230.15 monoclonal antibodies, were less efficiently protected but displayed significant reductions in weight loss that approached 12% weight by day 4 (t-test; p<0.01; FIG. 3C). In addition, all animals receiving S227.14 or S230.15 mAbs recovered by day 5. In contrast animals that received the irrelevant mAb D2.2 or S109.8 were not protected against weight loss after challenge with homologous or heterologous icSARS-CoV, e.g. all animals lost >20% by day 4 post infection (FIG. 3C). In addition, virus titers remained high in mice that received S109.8 and challenged with icUrbani or icGZ02, or in any of the BALB/c mice challenged with icHC/SZ/61/03, demonstrating that this antibody was less efficient at protecting animals from lethal infection especially at low dose (FIGS. 3D and E).

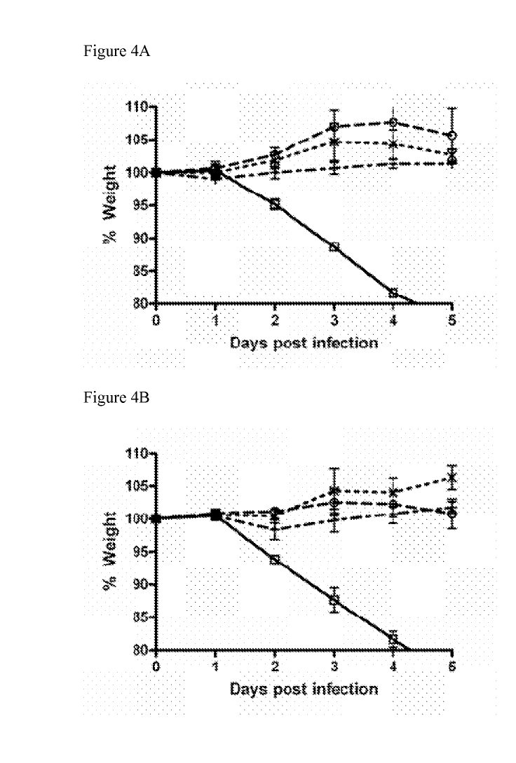

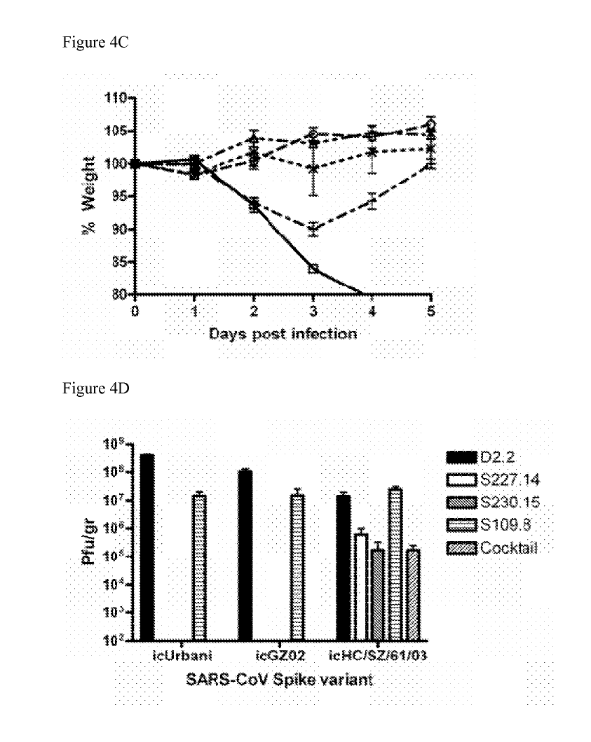

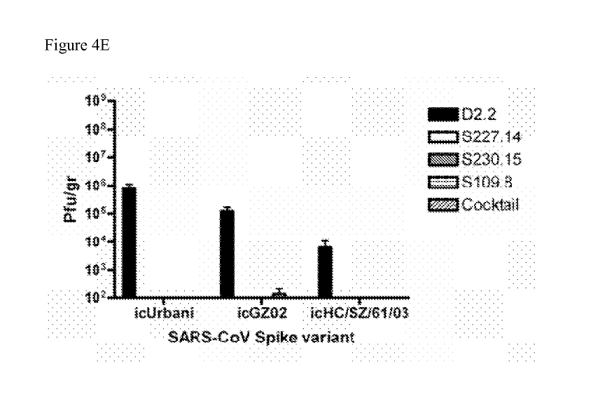

We used a very high dose of the challenge inocula to provide the most stringent test for mAb effectiveness, so it was not surprising that a 25 μg mAb dose produced variable results with some mAb and challenge viruses. To determine whether a high dose of mAb would enhance prophylaxis against clinical disease and death, 12-month-old BALB/c mice were dosed with 250 μg D2.2, S109.8, S227.14 or S230.15 one day prior to infection. As expected, animals that received S227.14 or S230.15, were protected against significant weight loss after challenge with icUrbani, icGZ02 or icHC/SZ/61/03 (t-test; p<0.01; FIGS. 4A, B and C). Importantly, the 10-fold increased dose of S109.8 was completely protective, as animals did not lose significant weight after challenge with icUrbani and icGZ02 and were partially protected against icHC/SZ/61/03 clinical disease with animals losing significantly less weight (˜10% weight by day 3, t-test; p<0.01) as compared to icUrbani challenged animals (FIGS. 4A, B and C). Importantly, animals recovered by day 5 post infection demonstrating that the antibody protected against severe clinical disease and death (FIG. 4C). No virus could be detected in lungs of animals that received S227.14 or S230.15 following challenge with icUrbani or icGZ02 on day 2 and 5 (ANOVA; p<0.01; FIGS. 4D and E), but interestingly, only a >1 or >2 log reduction were observed respectively after challenge with icHC/SZ/61/03 (ANOVA; p<0.05). In the lungs of BALB/c mice that received S109.8, only limited reduction of viral titers was observed (˜1 log) on day 2 post challenge with icUrbani or icGZ02 (ANOVA; p<0.01) and no reduction in icHC/SZ/61/03 titers. However, no viral replication could be detected in lungs infected with any of the viruses at day 5 post infection, demonstrating an enhanced rate of clearance over time (FIG. 4E).

### Example 9

Broad Spectrum Monoclonal Antibody Cocktail. Previous studies have suggested that cocktails of neutralizing antibodies may enhance protection against virus infection (48). Since single mAb treatment regimens did not protect 12-month-old BALB/c mice against virus replication after challenge with the heterologous icHC/SZ/61/03 strain, animals were dosed with a cocktail of equal amounts of the S109.8, S227.14 and S230.15 mAbs (83 μg of each mAb) at a final concentration of 250 μg; testing the hypothesis that multiple mAbs that recognized distinct neutralizing epitopes may increase immunization efficacy. Animals that received the cocktail were completely protected against weight loss following infection with icHC/SZ/61/03 (t-test; p<0.01; FIG. 4C). In addition, viral titers in the lungs on day 2 post challenge (FIG. 4D) were similar to those in animals that received the S227.14 or S230.15 mAbs alone, but about 2 log lower compared to animals that received the S109.8 mAb alone. As seen in mice treated with a single mAb, no virus could be detected at 5 days post infection (FIG. 4E).

### Example 10

Protection from Lethal Challenge in Young Mice. The S230.15 mAb has recently been shown to protect against replication of recombinant SARS-CoV bearing another palm civet S glycoprotein (SZ16) in young mice (39). Surprisingly, the same mAb did not completely protect 12-month-old BALB/c mice against lethal challenge with another civet variant, icHC/SZ/61/03. To determine whether the failure of the passive immunization against icHC/SZ/61/03 was specific for aged mice, an identical passive immunization experiment was performed in 10-week-old BALB/c mice. As shown previously in 8-week-old mice (39), young mice challenged with icHC/SZ/61/03 did not loose weight or display other clinical disease symptoms (data not shown) and virus titers in young and old mice were comparable. Interestingly only 1 out of 3 mice that received a dose of 250 μg S230.15 had detectable viral titers (7*10⁶ pfu/gr), demonstrating enhanced functional activity in younger animals. In control animals, icHCSZ6103 replicates to equivalent titers at day 2 post infection, suggesting that passive antibody transfer may be less efficient at protecting the lungs of immunosenescent populations.

The recent development of a mouse adapted SARS-CoV (icMA15) (35) allowed us to test mAb effectiveness in young mice against a homologous lethal challenge virus. The MA15 virus has a single mouse-adapted change in the S glycoprotein at residue Y436H. Ten-week-old BALB/c mice that received 25 μg of S227.14 were significantly and completely protected against weight loss after challenge with icMA15 (FIG. 5A). Animals that received either S230.15 or S109.8 all had significant weight loss starting by day 3 or 2 post infection respectively (t-test; p<0.01, with a maximum of 15%, but eventually leveled out by day 4 (FIG. 5A). Virus titers in lungs of animals that received S227.14 were lower on day 2 as compared to S230.15, S109.8 and the D2.2 control (FIG. 5B). Interestingly, at day 4, no virus could be detected in lungs of animals treated with S227.14 (FIG. 5C), suggesting that the icMA15 mutation Y436H affected S230.15 binding and neutralization efficacy.

### Example 11

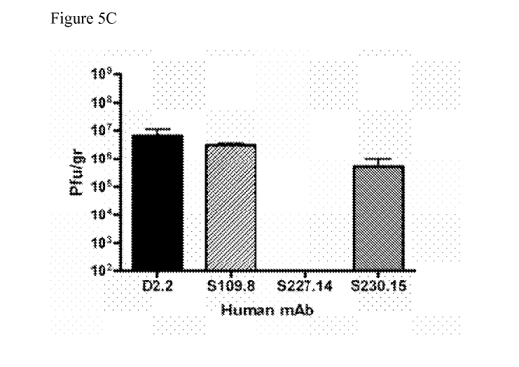

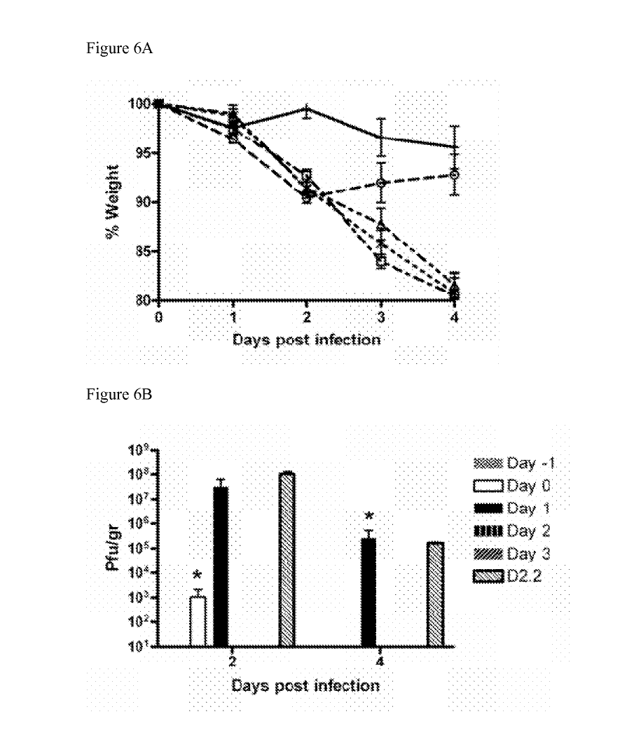

Post infection treatment of Lethal Challenge. Given the possibility of lethal infection and community spread, antibody prophylaxis following SARS-CoV exposure is an important Public Health consideration and especially for laboratory personnel. Therefore, one of the most efficient cross-neutralizing mAbs, S230.15 was used prophylactically at a dose of 250 μg at different times post-exposure with icGZ02 in an aged infection model. Complete protection from weight loss was observed when 12-month-old BALB/c mice were immunized 1 day prior to challenge (FIG. 6A). Mice immunized at the time of infection lost up to 10% weight by day 2 post challenge (t-test; p<0.01), but recovered by day 3. Treatment of BALB/c mice at 1, 2 or 3 days post challenge did not protect against weight loss suggesting a narrow window of prophylactic activity in the acute lethal mouse model. (FIG. 6A).

Virus titers were examined in the lungs on days 2 and 4. By day 2 post challenge, complete protection against virus replication in lungs of BALB/c mice treated with mAb 1 day prior to challenge was observed (ANOVA; p<0.01; FIG. 6B). In contrast, a 5 log reduction in virus titers was observed when treated on the day of challenge (detectable virus in only 1 out of 4 animals, ANOVA; p<0.01). Consonant with the development of severe clinical disease, no reduction in viral titers was observed when treated 1 day post challenge (FIG. 6B). By day 4 post challenge, virus was no longer detectable in lungs of mice treated −1, 0, 2 or 3 days post challenge (ANOVA; p<0.01) and only detectable in 1 out of 5 BALB/c mice treated with mAb on day 1 post challenge (FIG. 6B).

These data suggest that the lethal course of SARS-CoV infection in the mouse model may well be set within the first 24h post infection, as this mAb was not capable of reducing the clinical course of disease.

### Example 12

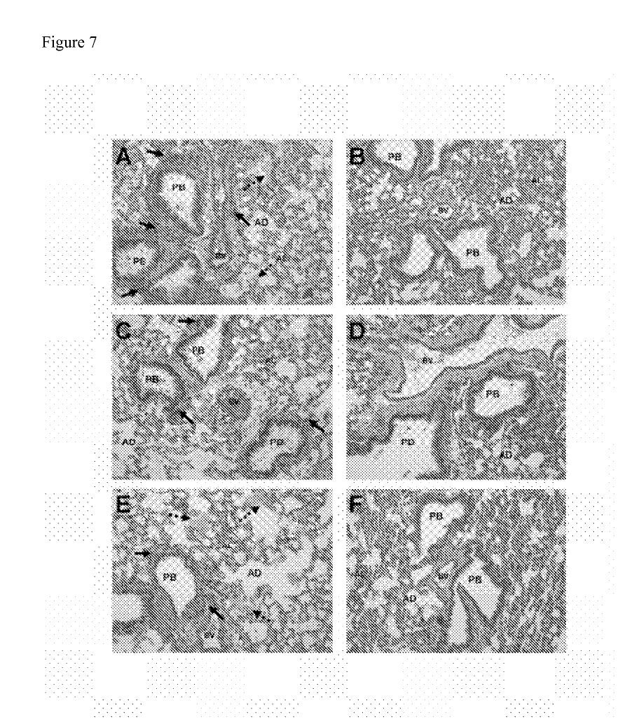

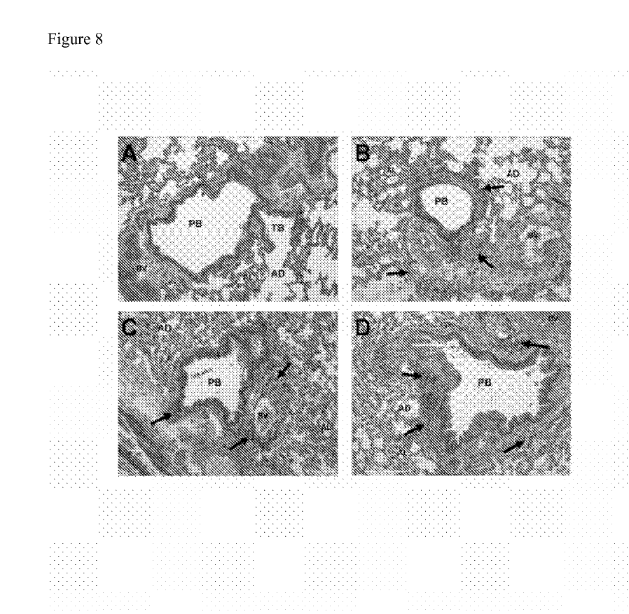

Pathologic Findings. The recapitulation in BALB/c mice, of the age-related pathology observed in acute case of SARS-CoV infection in humans (39), provides us with a third measure of protection along with morbidity and viral titers. Though there was some animal-to-animal variation, in general 12-month-old BALB/c mice that received the control mAb D2.2 showed evidence of bronchiolitis with epithelial cell exfoliation, virus-induced peribronchiolar inflammation, diffuse acute alveolitis and numerous hyaline membranes in the alveolar airspaces after infection with icUrbani (FIG. 7A), icGZ02 (FIG. 7C) and icHC/SZ/61/03 (FIG. 7E). Animals that received 250 μg of either mAbs or a cocktail of the three mAbs showed a marked decrease in bronchiolitis, exfoliation and alveolar inflammation and hyaline membrane formation was absent (FIGS. 7B, D and F). No clear decrease in alveolar inflammation and bronchiolitis was observed when animals received 25 μg of either mAb, however animals were protected against hyaline membrane formation (data not shown). Finally, in agreement with the morbidity data, post-infection treatment only showed a clear reduction in pathologic changes when treated one day prior to (FIG. 7D) or on the day of infection (FIG. 8A) but not on days 1, 2 or 3 post infection (FIGS. 8B, C and D respectively).

No evidence of enhanced disease or pathology was observed with any of the mAb and any of the challenge viruses.

### REFERENCES

1. Aw, D. et al, 2007. Immunosenescence: emerging challenges for an ageing population. Immunology 120:435-46.
2. Bakker, A. B. et al, 2005. Novel human monoclonal antibody combination effectively neutralizing natural rabies virus variants and individual in vitro escape mutants. J Virol 79:9062-8.
3. Baric, R. S. et al, 2006. SARS coronavirus vaccine development. Adv Exp Med Biol 581:553-60.
4. Chan-Yeung, M., and R. H. Xu. 2003. SARS: epidemiology. Respirology 8 Suppl:S9-14.
5. Chan, K. C., et al, 2005. Absence of association between angiotensin converting enzyme polymorphism and development of adult respiratory distress syndrome in patients with severe acute respiratory syndrome: a case control study. BMC Infect Dis 5:26.
6. Chinese, S. M. E. C. 2004. Molecular evolution of the SARS coronavirus during the course of the SARS epidemic in China. Science 303:1666-9.
7. Day, P. M., et al, 2007. Neutralization of human papillomavirus with monoclonal antibodies reveals different mechanisms of inhibition. J Virol 81:8784-92.
8. Deming, D., et al, 2006. Vaccine efficacy in senescent mice challenged with recombinant SARS-CoV bearing epidemic and zoonotic spike variants. PLoS Med 3:e525.
9. Dickinson, B. L., et al, 1999. Bidirectional FcRn-dependent IgG transport in a polarized human intestinal epithelial cell line. J Clin Invest 104:903-11.
10. Giachino, C., et al, 1995. kappa+lambda+dual receptor B cells are present in the human peripheral repertoire. J Exp Med 181:1245-50.
11. Greenough, T. C., et al, 2005. Development and characterization of a severe acute respiratory syndrome-associated coronavirus-neutralizing human monoclonal antibody that provides effective immunoprophylaxis in mice. J Infect Dis 191:507-14.
12. Guan, Y., B. et al, 2003. Isolation and characterization of viruses related to the SARS coronavirus from animals in southern China. Science 302:276-8.
13. He, Y., et al, 2006. Cross-neutralization of human and palm civet severe acute respiratory syndrome coronaviruses by antibodies targeting the receptor-binding domain of spike protein. J Immunol 176:6085-92.
14. He, Y., et al, 2004. Receptor-binding domain of SARS-CoV spike protein induces highly potent neutralizing antibodies: implication for developing subunit vaccine. Biochem Biophys Res Commun 324:773-81.
15. Huang, K. J., et al, 2005. An interferon-gamma-related cytokine storm in SARS patients. J Med Virol 75:185-94.
16. Hwang, W. C., et al, 2006. Structural basis of neutralization by a human anti-severe acute respiratory syndrome spike protein antibody, 80R. J Biol Chem 281:34610-6.
17. Kan, B., et al, 2005. Molecular evolution analysis and geographic investigation of severe acute respiratory syndrome coronavirus-like virus in palm civets at an animal market and on farms. J Virol 79:11892-900.
18. Kaverin, N. V., et al, 2002. Structure of antigenic sites on the haemagglutinin molecule of H5 avian influenza virus and phenotypic variation of escape mutants. J Gen Virol 83:2497-505.
19. Kim, S. J., et al, 2004. Neutralizing human monoclonal antibodies to hepatitis A virus recovered by phage display. Virology 318:598-607.
20. Klasse, P. J., and Q. J. Sattentau. 2002. Occupancy and mechanism in antibody-mediated neutralization of animal viruses. J Gen Virol 83:2091-108.
21. Kuiken, T., et al, 2003. Newly discovered coronavirus as the primary cause of severe acute respiratory syndrome. Lancet 362:263-70.
22. Lai, S. C., et al, 2005. Characterization of neutralizing monoclonal antibodies recognizing a 15-residues epitope on the spike protein HR2 region of severe acute respiratory syndrome coronavirus (SARS-CoV). J Biomed Sci 12:711-27.
23. Li, F., et al, 2005. Structure of SARS coronavirus spike receptor-binding domain complexed with receptor. Science 309:1864-8.
24. Li, W., et al, 2003. Angiotensin-converting enzyme 2 is a functional receptor for the SARS coronavirus. Nature 426:450-4.
25. Li, W., et al, 2005. Bats are natural reservoirs of SARS-like coronaviruses. Science 310:676-9.
26. Li, W., et al, 2005. Receptor and viral determinants of SARS-coronavirus adaptation to human ACE2. Embo J 24:1634-43.
27. Lim, P. L., et al, 2004. Laboratory-acquired severe acute respiratory syndrome. N Engl J Med 350:1740-5.
28. Liu, M., et al, 2006. Risk factors for SARS-related deaths in 2003, Beijing. Biomed Environ Sci 19:336-9.
29. Moore, J. P., and J. Sodroski. 1996. Antibody cross-competition analysis of the human immunodeficiency virus type 1 gp120 exterior envelope glycoprotein. J Virol 70:1863-72.
30. Nelson, J. D., et al, 2007. An affinity-enhanced neutralizing antibody against the membrane-proximal external region of human immunodeficiency virus type 1 gp41 recognizes an epitope between those of 2F5 and 4E10. J Virol 81:4033-43.
31. Nie, Y., et al, 2004. Neutralizing antibodies in patients with severe acute respiratory syndrome-associated coronavirus infection. J Infect Dis 190:1119-26.
32. Oliphant, T., et al, 2006. Antibody recognition and neutralization determinants on domains I and II of West Nile Virus envelope protein. J Virol 80:12149-59.
33. Orellana, C. 2004. Laboratory-acquired SARS raises worries on biosafety. Lancet Infect Dis 4:64.
34. Prabakaran, P., et al, 2006. Structure of severe acute respiratory syndrome coronavirus receptor-binding domain complexed with neutralizing antibody. J Biol Chem 281:15829-36.
35. Roberts, A., et al, 2007. A mouse-adapted SARS-coronavirus causes disease and mortality in BALB/c mice. PLoS Pathog 3:e5.
36. Roberts, A., et al, 2005. Aged BALB/c mice as a model for increased severity of severe acute respiratory syndrome in elderly humans. J Virol 79:5833-8.
37. Roberts, A., et al, 2006. Therapy with a severe acute respiratory syndrome-associated coronavirus-neutralizing human monoclonal antibody reduces disease severity and viral burden in golden Syrian hamsters. J Infect Dis 193:685-92.
38. Rockx, B., and R. S. Baric. 2007. Grand Challenges in Human Coronavirus Vaccine Development. Coronaviruses: Molecular and Cellular Biology.
39. Rockx, B., et al, 2007. Synthetic reconstruction of zoonotic and early human severe acute respiratory syndrome coronavirus isolates that produce fatal disease in aged mice. J Virol 81:7410-23.
40. Roohi, A., et al, 2005. Epitope mapping of recombinant hepatitis B surface antigen by murine monoclonal antibodies. Hybridoma (Larchmt) 24:71-7.
41. Sawyer, L. A. 2000. Antibodies for the prevention and treatment of viral diseases. Antiviral Res 47:57-77.
42. Sheng, W. H., et al, 2005. Clinical manifestations and inflammatory cytokine responses in patients with severe acute respiratory syndrome. J Formos Med Assoc 104:715-23.
43. Simmons, C. P., et al, 2007. Prophylactic and therapeutic efficacy of human monoclonal antibodies against H5N1 influenza. PLoS Med 4:e178.
44. Subbarao, K., et al, 2004. Prior infection and passive transfer of neutralizing antibody prevent replication of severe acute respiratory syndrome coronavirus in the respiratory tract of mice. J Virol 78:3572-7.
45. Sui, J., et al, 2005. Evaluation of human monoclonal antibody 80R for immunoprophylaxis of severe acute respiratory syndrome by an animal study, epitope mapping, and analysis of spike variants. J Virol 79:5900-6.
46. Targonski, P. V., et al, 2007. Immunosenescence: role and measurement in influenza vaccine response among the elderly. Vaccine 25:3066-9.
47. ter Meulen, J., et al, 2004. Human monoclonal antibody as prophylaxis for SARS coronavirus infection in ferrets. Lancet 363:2139-41.
48. ter Meulen, J., et al, 2006. Human monoclonal antibody combination against SARS coronavirus: synergy and coverage of escape mutants. PLoS Med 3:e237.
49. Traggiai, E., et al, 2004. An efficient method to make human monoclonal antibodies from memory B cells: potent neutralization of SARS coronavirus. Nat Med 10:871-5.
50. Tripp, R. A., et al, 2005. Monoclonal antibodies to SARS-associated coronavirus (SARS-CoV): identification of neutralizing and antibodies reactive to S, N, M and E viral proteins. J Virol Methods 128:21-8.
51. Tzartos, S. J. 1996. Epitope mapping by antibody competition. Methodology and Evaluation of the validity of the technique. Methods Mol Bio1:55-66.
52. Vicenzi, E., et al, 2004. Coronaviridae and SARS-associated coronavirus strain HSR1. Emerg Infect Dis 10:413-8.
53. Vogel, L. N., et al, 2007. Utility of the aged BALB/c mouse model to demonstrate prevention and control strategies for severe acute respiratory syndrome coronavirus (SARS-CoV). Vaccine 25:2173-9.
54. Wang, P., et al, 2004. Expression cloning of functional receptor used by SARS coronavirus. Biochem Biophys Res Commun 315:439-44.
55. Wien, M. W., et al, 1995. Structure of the complex between the Fab fragment of a neutralizing antibody for type 1 poliovirus and its viral epitope. Nat Struct Biol 2:232-43.
56. Yang, X., et al, 2007. Antibody Binding in Proximity to the Receptor/Glycoprotein Complex Leads to, a Basal Level of Virus Neutralization. J Virol.
57. Yang, Z. Y., et al, 2004. pH-dependent entry of severe acute respiratory syndrome coronavirus is mediated by the spike glycoprotein and enhanced by dendritic cell transfer through DC-SIGN. J Virol 78:5642-50.
58. Yang, Z. Y., et al, 2005. Evasion of antibody neutralization in emerging severe acute respiratory syndrome coronaviruses. Proc Natl Acad Sci USA 102:797-801.
59. Zhao, G. P. 2007. SARS epidemiology-From descriptive to mechanistic analyses. Virus Res.
60. Zhu, Z., et al, 2007. Potent cross-reactive neutralization of SARS coronavirus isolates by human monoclonal antibodies. Proc Natl Acad Sci USA.

We claim:

1. A monoclonal antibody that cross neutralizes at least three strains of SARS-CoV.

2. The monoclonal antibody of claim 1 wherein said monoclonal antibody cross neutralizes at least five strains of SARS-CoV.

3. The monoclonal antibody of claim 1 or claim 2 wherein said strains of SARS-CoV are selected from the group consisting of Urbani, CUHK-W, GZ02, HC/SZ/61/03, and A031G.

4. The monoclonal antibody of claim 1, wherein said monoclonal antibody binds to an epitope that comprises an amino acid at positions 332, 333, 390, 436, 443, or 487 of the SARS-CoV Spike protein.

5. The monoclonal antibody of claim 1, wherein said monoclonal antibody binds to an epitope that comprises at least 2 amino acids at positions 332, 333, 390, 436, 443, or 487 of the SARS-CoV Spike protein.

6. The monoclonal antibody of claim 1, wherein said monoclonal antibody binds to an epitope that comprises amino acids at positions 332 and 333 of the SARS-CoV Spike protein.

7. The monoclonal antibody of claim 1, wherein said monoclonal antibody binds to an epitope that comprises at least 3 amino acids at positions 332, 333, 390, 436, 443, or 487 of the SARS-CoV Spike protein.

8. The monoclonal antibody of claim 1, wherein said monoclonal antibody binds to an epitope that comprises amino acids at positions 436, 443 and 487 of the SARS-CoV Spike protein.

9. The monoclonal antibody according to claim 1, wherein the antibody has an affinity of 10⁻⁸M or less for the SARS-CoV Spike protein.

10. The monoclonal antibody of claim 1, wherein said monoclonal antibody has a 50% neutralization concentration of less than 1 μg/ml.

11. A monoclonal antibody which neutralizes SARS-CoV, wherein said antibody has a light chain with three CDRs, each comprising an amino acid sequence selected from the group consisting of the amino acid sequences of SEQ ID NOs: 52-60.

12. A monoclonal antibody which neutralizes SARS-CoV, wherein said antibody has a heavy chain with three CDRs, each comprising an amino acid sequence selected from the group consisting of the amino acid sequences of SEQ ID NOs: 22-30.

13. A monoclonal antibody which neutralizes SARS-CoV, wherein said antibody has an amino acid sequence comprising any one of the following pairs:
SEQ ID NOs: 90 and 92;
SEQ ID NOs: 94 and 96; or
SEQ ID NOs: 98 and 101.

14. A monoclonal antibody that binds to an epitope which is bound by the antibody of any one of claims 1-13, or an antibody that competes with the antibody of any one of claims 1-13.

15. The monoclonal antibody of any one of claims 1-13, wherein neutralization ability of said monoclonal antibody is decreased by a mutation at positions 332, 333, 390, 436, 443, or 487 of SARS-CoV spike protein.

16. The monoclonal antibody of any one of claims 1-13, wherein the monoclonal antibody is S109.8, S227.14 or S230.15.

17. A method of preventing a disease or disorder caused by a coronavirus, the method comprising:
administering to a person at risk of, or suffering from, said disease or disorder a therapeutically effective amount of the monoclonal antibody of any one of claims 1-13.
